SEQUENCE LISTING (27 sequences)

SEQ ID NO: 1 - DNA, 2657 bp, Peptostreptococcus magnus
  CDS (301)..(2460): protein L
  repeat_unit (592)..(819): PpL1
  repeat_unit (820)..(1035): PpL2
  repeat_unit (1036)..(1251): PpL3
  repeat_unit (1252)..(1467): PpL4
  repeat_unit (1468)..(1686): PpL5
atagtgctacaataaaggatggcactgaactaccgaacctaacgcttgtggacattgtcttgggaaatttggacagtggacgaatcaagaacaccattaattaaattggtgaagttcgattgttgatcaccctttttgggtaaacaataactaaggaatggcaatatatttgcttggaaacgaatttgatttaaatagcattgaatgcaaaaaaatttaaaaggaggagacaaattccacccttattagaagggaagtttccattgtcatgatattatgaaaattaataagaaattattaatggctgcacttgcaggtgcaattgtagtaacaggtggagtaggatcttacgcagctgatgaacctattgatcttgaaaaacttgaagaaaaaagggataaagaaaatgtaggaaatttaccaaaattcgataatgaagttaaagatggttcagaaaatccaatggctaaatatccagatttcgatgatgaagccagtacaagatttgaaacagaaaacaatgaatttgaagaaaaaaaagttgtttctgataacttttttgatcaatcagaacatccgtttgtagaaaataaagaagaaacaccagaaacaccagaaactgattcagaagaagaagtaacaatcaaagctaacctaatctttgcaaatggaagcacacaaactgcagaattcaaaggaacatttgaaaaagcaacatcagaagcttatgcgtatgcagatactttgaagaaagacaatggagaatatactgtagatgttgcagataaaggttatactttaaatattaaatttgctggaaaagaaaaaacaccagaagaaccaaaagaagaagttactattaaagcaaacttaatctatgcagatggaaaaacacaaacagcagaattcaaaggaacatttgaagaagcaacagcagaagcatacagatatgcagatgcattaaagaaggacaatggagaatatacagtagacgttgcagataaaggttatactttaaatattaaatttgctggaaaagaaaaaacaccagaagaaccaaaagaagaagttactattaaagcaaacttaatctatgcagatggaaaaacacaaacagcagaattcaaaggaacatttgaagaagcaacagcagaagcatacagatatgctgacttattagcaaaagaaaatggtaaatatacagtagacgttgcagataaaggttatactttaaatattaaatttgctggaaaagaaaaaacaccagaagaaccaaaagaagaagttactattaaagcaaacttaatctatgcagatggaaaaactcaaacagcagagttcaaaggaacatttgcagaagcaacagcagaagcatacagatacgctgacttattagcaaaagaaaatggtaaatatacagcagacttagaagatggtggatacactattaatattagatttgcaggtaagaaagttgacgaaaaaccagaagaaaaagaacaagtaacaattaaagaaaatatatattttgaagatggaacagtacaaactgcaacatttaaaggaacatttgcagaagcgacagcagaagcatacagatatgcagatttgttatcaaaagaacatggtaaatacacagcagacttggaagatggtggatacactatcaacattagatttgctggaaaagaagaaccagaagaaacaccagaaaaaccagaagtacaagacggatatgcatcatacgaagaagctgaagcagcagctaaagaagctttgaaaaatgatgatgtaaataaatcatatactattagacaaggtgcagatggaagatattactatgtattatcaccagtagaagctgaagaagaaaaaccagaagcacaaaatggatatgcaacatacgaagaagcagaagcagcagctaagaaagctttggaaaatgatccaatcaataaatcttacagcataagacaaggtgcagatggaagatactactatgtattatcaccagtagaagctgaaactcctgaaaaaccagtagaaccatcagaaccatcaacaccagatgtaccatcaaacccatcaaatccatcaacaccagatgttccatcaactcctgatgtaccatcaaatccttcaactccagaagttccttcaaacccatcaactcctggtaatgaagaaaaacctggtaacgaacaaaaacctggtaacgaacaaaaacctggtaacgaacaaaaacctggtaatgaacaaaaacctggtaatgaacaaaaaccagaccaaccttcaaaaccagaaaaagaagaaaatggtaagggtggagtagattctccaaagaaaaaagaaaaagctgcattaccaaaagctggtagcgaagctgaaatcttaacattagcagcagcttcattatcaagcgttgcaggtgctttcatttcacttaaaaaacgtaaataattaatcttagataaaaaatagattaatctaaaaaaatgggacttttataagtcccatttttgattgcaatgaaactgatacaaaaaatgtatcagttttttcatttacgcttattttcctgtgagtatgtccaagtttgtcgtataatcatctatcaaaagcctggcgagtttcgctttttggattttgtcttcgaa

SEQ ID NO: 2 - protein, 719 aa, Peptostreptococcus magnus
MAALAGAIVVTGGVGSYAADEPIDLEKLEEKRDKENVGNLPKFDNEVKDGSENPMAKYPDFDDEASTRFETENNEFEEKKVVSDNFFDQSEHPFVENKEETPETPETDSEEEVTIKANLIFANGSTQTAEFKGTFEKATSEAYAYADTLKKDNGEYTVDVADKGYTLNIKFAGKEKTPEEPKEEVTIKANLIYADGKTQTAEFKGTFEEATAEAYRYADALKKDNGEYTVDVADKGYTLNIKFAGKEKTPEEPKEEVTIKANLIYADGKTQTAEFKGTFEEATAEAYRYADLLAKENGKYTVDVADKGYTLNIKFAGKEKTPEEPKEEVTIKANLIYADGKTQTAEFKGTFAEATAEAYRYADLLAKENGKYTADLEDGGYTINIRFAGKKVDEKPEEKEQVTIKENIYFEDGTVQTATFKGTFAEATAEAYRYADLLSKEHGKYTADLEDGGYTINIRFAGKEEPEETPEKPEVQDGYASYEEAEAAAKEALKNDDVNKSYTIRQGADGRYYYVLSPVEAEEEKPEAQNGYATYEEAEAAAKKALENDPINKSYSIRQGADGRYYYVLSPVEAETPEKPVEPSEPSTPDVPSNPSNPSTPDVPSTPDVPSNPSTPEVPSNPSTPGNEEKPGNEQKPGNEQKPGNEQKPGNEQKPGNEQKPDQPSKPEKEENGKGGVDSPKKKEKAALPKAGSEAEILTLAAASLSSVAGAFISLKKRK

SEQ ID NO: 3 - DNA, 228 bp, Peptostreptococcus magnus
  CDS (1)..(228): PpL1
aaagaagaaacaccagaaacaccagaaactgattcagaagaagaagtaacaatcaaagctaacctaatctttgcaaatggaagcacacaaactgcagaattcaaaggaacatttgaaaaagcaacatcagaagcttatgcgtatgcagatactttgaagaaagacaatggagaatatactgtagatgttgcagataaaggttatactttaaatattaaatttgctgga

SEQ ID NO: 4 - protein, 76 aa, Peptostreptococcus magnus
KEETPETPETDSEEEVTIKANLIFANGSTQTAEFKGTFEKATSEAYAYADTLKKDNGEYTVDVADKGYTLNIKFAG

SEQ ID NO: 5 - DNA, 216 bp, Peptostreptococcus magnus
  CDS (1)..(216): PpL2
aaagaaaaaacaccagaagaaccaaaagaagaagttactattaaagcaaacttaatctatgcagatggaaaaacacaaacagcagaattcaaaggaacatttgaagaagcaacagcagaagcatacagatatgcagatgcattaaagaaggacaatggagaatatacagtagacgttgcagataaaggttatactttaaatattaaatttgctgga

SEQ ID NO: 6 - protein, 72 aa, Peptostreptococcus magnus
KEKTPEEPKEEVTIKANLIYADGKTQTAEFKGTFEEATAEAYRYADALKKDNGEYTVDVADKGYTLNIKFAG

SEQ ID NO: 7 - DNA, 216 bp, Peptostreptococcus magnus
  CDS (1)..(216): PpL3
aaagaaaaaacaccagaagaaccaaaagaagaagttactattaaagcaaacttaatctatgcagatggaaaaacacaaacagcagaattcaaaggaacatttgaagaagcaacagcagaagcatacagatatgctgacttattagcaaaagaaaatggtaaatatacagtagacgttgcagataaaggttatactttaaatattaaatttgctgga

SEQ ID NO: 8 - protein, 72 aa, Peptostreptococcus magnus
KEKTPEEPKEEVTIKANLIYADGKTQTAEFKGTFEEATAEAYRYADLLAKENGKYTVDVADKGYTLNIKFAG

SEQ ID NO: 9 - DNA, 216 bp, Peptostreptococcus magnus
  CDS (1)..(216): PpL4
aaagaaaaaacaccagaagaaccaaaagaagaagttactattaaagcaaacttaatctatgcagatggaaaaactcaaacagcagagttcaaaggaacatttgcagaagcaacagcagaagcatacagatacgctgacttattagcaaaagaaaatggtaaatatacagcagacttagaagatggtggatacactattaatattagatttgcaggt

SEQ ID NO: 10 - protein, 72 aa, Peptostreptococcus magnus
KEKTPEEPKEEVTIKANLIYADGKTQTAEFKGTFAEATAEAYRYADLLAKENGKYTADLEDGGYTINIRFAG

SEQ ID NO: 11 - DNA, 219 bp, Peptostreptococcus magnus
  CDS (1)..(219): PpL5
aagaaagttgacgaaaaaccagaagaaaaagaacaagtaacaattaaagaaaatatatattttgaagatggaacagtacaaactgcaacatttaaaggaacatttgcagaagcgacagcagaagcatacagatatgcagatttgttatcaaaagaacatggtaaatacacagcagacttggaagatggtggatacactatcaacattagatttgctgga

SEQ ID NO: 12 - protein, 73 aa, Peptostreptococcus magnus
KKVDEKPEEKEQVTIKENIYFEDGTVQTATFKGTFAEATAEAYRYADLLSKEHGKYTADLEDGGYTINIRFAG

SEQ ID NO: 13 - DNA, 1269 bp, Sindbis virus
  misc_feature (1)..(1269): E2 potein gene from strain AR339
  CDS (1)..(1269): E2 glycoprotein
  variation (3)..(3): a in AR339; c in HRsp
  variation (8)..(8): c in AR339; t in HRsp
  variation (68)..(68): a in AR339; t in HRsp
  variation (208)..(208): g in AR339; a in HRsp
  variation (514)..(514): g in AR339; a in HRsp
agagtcactgacgactttaccctgaccagcccctacttgggcacatgctcgtactgccaccatactgaaccgtgcttcagccctgttaagatcgagcaggtctgggacgaagcggacgataacaccatacgcatacagacttccgcccagtttggatacgaccaaagcggagcagcaagcgcaaacaagtaccgctacatgtcgcttgagcaggatcacaccgttaaagaaggcaccatggatgacatcaagattagcacctcaggaccgtgtagaaggcttagctacaaaggatactttctcctcgcaaaatgccctccaggggacagcgtaacggttagcatagtgagtagcaactcagcaacgtcatgtacactggcccgcaagataaaaccaaaattcgtgggacgggaaaaatatgatctacctcccgttcacggtaaaaaaattccttgcacagtgtacgaccgtctgaaagaaacaactgcaggctacatcactatgcacaggccgggaccgcacgcttatacatcctacctggaagaatcatcagggaaagtttacgcaaagccgccatctgggaagaacattacgtatgagtgcaagtgcggcgactacaagaccggaaccgtttcgacccgcaccgaaatcactggttgcaccgccatcaagcagtgcgtcgcctataagagcgaccaaacgaagtgggtcttcaactcaccggacttgatcagacatgacgaccacacggcccaagggaaattgcatttgcctttcaagttgatcccgagtacctgcatggtccctgttgcccacgcgccgaatgtaatacatggctttaaacacatcagcctccaattagatacagaccacttgacattgctcaccaccaggagactaggggcaaacccggaaccaaccactgaatggatcgtcggaaagacggtcagaaacttcaccgtcgaccgagatggcctggaatacatatggggaaatcatgagccagtgagggtctatgcccaagagtcagcaccaggagaccctcacggatggccacacgaaatagtacagcattactaccatcgccatcctgtgtacaccatcttagccgtcgcatcagctaccgtggcgatgatgattggcgtaaccgttgcagtgttatgtgcctgtaaagcgcgccgtgagtgcctgacgccatacgccctggccccaaacgccgtaatcccaacttcgctggcactcttgtgctgcgttaggtcggccaatgct

SEQ ID NO: 14 - protein, 423 aa, Sindbis virus
RVTDDFTLTSPYLGTCSYCHHTEPCFSPVKIEQVWDEADDNTIRIQTSAQFGYDQSGAASANKYRYMSLEQDHTVKEGTMDDIKISTSGPCRRLSYKGYFLLAKCPPGDSVTVSIVSSNSATSCTLARKIKPKFVGREKYDLPPVHGKKIPCTVYDRLKETTAGYITMHRPGPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANA

SEQ ID NO: 15 - DNA, 11703 bp, Sindbis virus
  misc_feature (1)..(11703): Sindbis virus HRsp and wild-type strains complete genome
  CDS (8439)..(8630): E3 protein
  CDS (8631)..(9899): E2 protein
attgacggcgtagtacacactattgaatcaaacagccgaccaattgcactaccatcacaatggagaagccagtagtaaacgtagacgtagacccccagagtccgtttgtcgtgcaactgcaaaaaagcttcccgcaatttgaggtagtagcacagcaggtcactccaaatgaccatgctaatgccagagcattttcgcatctggccagtaaactaatcgagctggaggttcctaccacagcgacgatcttggacataggcagcgcaccggctcgtagaatgttttccgagcaccagtatcattgtgtctgccccatgcgtagtccagaagacccggaccgcatgatgaaatacgccagtaaactggcggaaaaagcgtgcaagattacaaacaagaacttgcatgagaagattaaggatctccggaccgtacttgatacgccggatgctgaaacaccatcgctctgctttcacaacgatgttacctgcaacatgcgtgccgaatattccgtcatgcaggacgtgtatatcaacgctcccggaactatctatcatcaggctatgaaaggcgtgcggaccctgtactggattggcttcgacaccacccagttcatgttctcggctatggcaggttcgtaccctgcgtacaacaccaactgggccgacgagaaagtccttgaagcgcgtaacatcggactttgcagcacaaagctgagtgaaggtaggacaggaaaattgtcgataatgaggaagaaggagttgaagcccgggtcgcgggtttatttctccgtaggatcgacactttatccagaacacagagccagcttgcagagctggcatcttccatcggtgttccacttgaatggaaagcagtcgtacacttgccgctgtgatacagtggtgagttgcgaaggctacgtagtgaagaaaatcaccatcagtcccgggatcacgggagaaaccgtgggatacgcggttacacacaatagcgagggcttcttgctatgcaaagttactgacacagtaaaaggagaacgggtatcgttccctgtgtgcacgtacatcccggccaccatatgcgatcagatgactggtataatggccacggatatatcacctgacgatgcacaaaaacttctggttgggctcaaccagcgaattgtcattaacggtaggactaacaggaacaccaacaccatgcaaaattaccttctgccgatcatagcacaagggttcagcaaatgggctaaggagcgcaaggatgatcttgataacgagaaaatgctgggtactagagaacgcaagcttacgtatggctgcttgtgggcgtttcgcactaagaaagtacattcgttttatcgcccacctggaacgcagacctgcgtaaaagtcccagcctcttttagcgcttttcccatgtcgtccgtatggacgacctctttgcccatgtcgctgaggcagaaattgaaactggcattgcaaccaaagaaggaggaaaaactgctgcaggtctcggaggaattagtcatggaggccaaggctgcttttgaggatgctcaggaggaagccagagcggagaagctccgagaagcacttccaccattagtggcagacaaaggcatcgaggcagccgcagaagttgtctgcgaagtggaggggctccaggcggacatcggagcagcattagttgaaaccccgcgcggtcacgtaaggataatacctcaagcaaatgaccgtatgatcggacagtatatcgttgtctcgccaaactctgtgctgaagaatgccaaactcgcaccagcgcacccgctagcagatcaggttaagatcataacacactccggaagatcaggaaggtacgcggtcgaaccatacgacgctaaagtactgatgccagcaggaggtgccgtaccatggccagaattcctagcactgagtgagagcgccacgttagtgtacaacgaaagagagtttgtgaaccgcaaactataccacattgccatgcatggccccgccaagaatacagaagaggagcagtacaaggttacaaaggcagagcttgcagaaacagagtacgtgtttgacgtggacaagaagcgttgcgttaagaaggaagaagcctcaggtctggtcctctcgggagaactgaccaaccctccctatcatgagctagctctggagggactgaagacccgacctgcggtcccgtacaaggtcgaaacaataggagtgataggcacaccggggtcgggcaagtcagctattatcaagtcaactgtcacggcacgagatcttgttaccagcggaaagaaagaaaattgtcgcgaaattgaggccgacgtgctaagactgaggggtatgcagattacgtcgaagacagtagattcggttatgctcaacggatgccacaaagccgtagaagtgctgtacgttgacgaagcgttcgcgtgccacgcaggagcactacttgccttgattgctatcgtcaggccccgcaagaaggtagtactatgcggagaccccatgcaatgcggattcttcaacatgatgcaactaaaggtacatttcaatcaccctgaaaaagacatatgcaccaagacattctacaagtatatctcccggcgttgcacacagccagttacagctattgtatcgacactgcattacgatggaaagatgaaaaccacgaacccgtgcaagaagaacattgaaatcgatattacaggggccacaaagccgaagccaggggatatcatcctgacatgtttccgcgggtgggttaagcaattgcaaatcgactatcccggacatgaagtaatgacagccgcggcctcacaagggctaaccagaaaaggagtgtatgccgtccggcaaaaagtcaatgaaaacccactgtacgcgatcacatcagagcatgtgaacgtgttgctcacccgcactgaggacaggctagtgtggaaaaccttgcagggcgacccatggattaagcagcccactaacatacctaaaggaaactttcaggctactatagaggactgggaagctgaacacaagggaataattgctgcaataaacagccccactccccgtgccaatccgttcagctgcaagaccaacgtttgctgggcgaaagcattggaaccgatactagccacggccggtatcgtacttaccggttgccagtggagcgaactgttcccacagtttgcggatgacaaaccacattcggccatttacgccttagacgtaatttgcattaagtttttcggcatggacttgacaagcggactgttttctaaacagagcatcccactaacgtaccatcccgccgattcagcgaggccggtagctcattgggacaacagcccaggaacccgcaagtatgggtacgatcacgccattgccgccgaactctcccgtagatttccggtgttccagctagctgggaagggcacacaacttgatttgcagacggggagaaccagagttatctctgcacagcataacctggtcccggtgaaccgcaatcttcctcacgccttagtccccgagtacaaggagaagcaacccggcccggtcaaaaaattcttgaaccagttcaaacaccactcagtacttgtggtatcagaggaaaaaattgaagctccccgtaagagaatcgaatggatcgccccgattggcatagccggtgcagataagaactacaacctggctttcgggtttccgccgcaggcacggtacgacctggtgttcatcaacattggaactaaatacagaaaccaccactttcagcagtgcgaagaccatgcggcgaccttaaaaaccctttcgcgttcggccctgaattgccttaacccaggaggcaccctcgtggtgaagtcctatggctacgccgaccgcaacagtgaggacgtagtcaccgctcttgccagaaagtttgtcagggtgtctgcagcgagaccagattgtgtctcaagcaatacagaaatgtacctgattttccgacaactagacaacagccgtacacggcaattcaccccgcaccatctgaattgcgtgatttcgtccgtgtatgagggtacaagagatggagttggagccgcgccgtcataccgcaccaaaagggagaatattgctgactgtcaagaggaagcagttgtcaacgcagccaatccgctgggtagaccaggcgaaggagtctgccgtgccatctataaacgttggccgaccagttttaccgattcagccacggagacaggcaccgcaagaatgactgtgtgcctaggaaagaaagtgatccacgcggtcggccctgatttccggaagcacccagaagcagaagccttgaaattgctacaaaacgcctaccatgcagtggcagacttagtaaatgaacataacatcaagtctgtcgccattccactgctatctacaggcatttacgcagccggaaaagaccgccttgaagtatcacttaactgcttgacaaccgcgctagacagaactgacgcggacgtaaccatctattgcctggataagaagtggaaggaaagaatcgacgcggcactccaacttaaggagtctgtaacagagctgaaggatgaagatatggagatcgacgatgagttagtatggatccatccagacagttgcttgaagggaagaaagggattcagtactacaaaaggaaaattgtattcgtacttcgaaggcaccaaattccatcaagcagcaaaagacatggcggagataaaggtcctgttccctaatgaccaggaaagtaatgaacaactgtgtgcctacatattgggtgagaccatggaagcaatccgcgaaaagtgcccggtcgaccataacccgtcgtctagcccgcccaaaacgttgccgtgcctttgcatgtatgccatgacgccagaaagggtccacagacttagaagcaataacgtcaaagaagttacagtatgctcctccaccccccttcctaagcacaaaattaagaatgttcagaaggttcagtgcacgaaagtagtcctgtttaatccgcacactcccgcattcgttcccgcccgtaagtacatagaagtgccagaacagcctaccgctcctcctgcacaggccgaggaggcccccgaagttgtagcgacaccgtcaccatctacagctgataacacctcgcttgatgtcacagacatctcactggatatggatgacagtagcgaaggctcacttttttcgagctttagcggatcggacaactctattactagtatggacagttggtcgtcaggacctagttcactagagatagtagaccgaaggcaggtggtggtggctgacgttcatgccgtccaagagcctgcccctattccaccgccaaggctaaagaagatggcccgcctggcagcggcaagaaaagagcccactccaccggcaagcaatagctctgagtccctccacctctcttttggtggggtatccatgtccctcggatcaattttcgacggagagacggcccgccaggcagcggtacaacccctggcaacaggccccacggatgtgcctatgtctttcggatcgttttccgacggagagattgatgagctgagccgcagagtaactgagtccgaacccgtcctgtttggatcatttgaaccgggcgaagtgaactcaattatatcgtcccgatcagccgtatcttttccactacgcaagcagagacgtagacgcaggagcaggaggactgaatactgactaaccggggtaggtgggtacatattttcgacggacacaggccctgggcacttgcaaaagaagtccgttctgcagaaccagcttacagaaccgaccttggagcgcaatgtcctggaaagaattcatgccccggtgctcgacacgtcgaaagaggaacaactcaaactcaggtaccagatgatgcccaccgaagccaacaaaagtaggtaccagtctcgtaaagtagaaaatcagaaagccataaccactgagcgactactgtcaggactacgactgtataactctgccacagatcagccagaatgctataagatcacctatccgaaaccattgtactccagtagcgtaccggcgaactactccgatccacagttcgctgtagctgtctgtaacaactatctgcatgagaactatccgacagtagcatcttatcagattactgacgagtacgatgcttacttggatatggtagacgggacagtcgcctgcctggatactgcaaccttctgccccgctaagcttagaagttacccgaaaaaacatgagtatagagccccgaatatccgcagtgcggttccatcagcgatgcagaacacgctacaaaatgtgctcattgccgcaactaaaagaaattgcaacgtcacgcagatgcgtgaactgccaacactggactcagcgacattcaatgtcgaatgctttcgaaaatatgcatgtaatgacgagtattgggaggagttcgctcggaagccaattaggattaccactgagtttgtcaccgcatatgtagctagactgaaaggccctaaggccgccgcactatttgcaaagacgtataatttggtcccattgcaagaagtgcctatggatagattcgtcatggacatgaaaagagacgtgaaagttacaccaggcacgaaacacacagaagaaagaccgaaagtacaagtgatacaagccgcagaacccctggcgactgcttacttatgcgggattcaccgggaattagtgcgtaggcttacggccgtcttgcttccaaacattcacacgctttttgacatgtcggcggaggattttgatgcaatcatagcagaacacttcaagcaaggcgacccggtactggagacggatatcgcatcattcgacaaaagccaagacgacgctatggcgttaaccggtctgatgatcttggaggacctgggtgtggatcaaccactactcgacttgatcgagtgcgcctttggagaaatatcatccacccatctacctacgggtactcgttttaaattcggggcgatgatgaaatccggaatgttcctcacactttttgtcaacacagttttgaatgtcgttatcgccagcagagtactagaagagcggcttaaaacgtccagatgtgcagcgttcattggcgacgacaacatcatacatggagtagtatctgacaaagaaatggctgagaggtgcgccacctggctcaacatggaggttaagatcatcgacgcagtcatcggtgagagaccaccttacttctgcggcggatttatcttgcaagattcggttacttccacagcgtgccgcgtggcggatcccctgaaaaggctgtttaagttgggtaaaccgctcccagccgacgacgagcaagacgaagacagaagacgcgctctgctagatgaaacaaaggcgtggtttagagtaggtataacaggcactttagcagtggccgtgacgacccggtatgaggtagacaatattacacctgtcctactggcattgagaacttttgcccagagcaaaagagcattccaagccatcagaggggaaataaagcatctctacggtggtcctaaatagtcagcatagtacatttcatctgactaatactacaacaccaccaccatgaatagaggattctttaacatgctcggccgccgccccttcccggcccccactgccatgtggaggccgcggagaaggaggcaggcggccccgatgcctgcccgcaacgggctggcttctcaaatccagcaactgaccacagccgtcagtgccctagtcattggacaggcaactagacctcaacccccacgtccacgcccgccaccgcgccagaagaagcaggcgcccaagcaaccaccgaagccgaagaaaccaaaaacgcaggagaagaagaagaagcaacctgcaaaacccaaacccggaaagagacagcgcatggcacttaagttggaggccgacagattgttcgacgtcaagaacgaggacggagatgtcatcgggcacgcactggccatggaaggaaaggtaatgaaacctctgcacgtgaaaggaaccatcgaccaccctgtgctatcaaagctcaaatttaccaagtcgtcagcatacgacatggagttcgcacagttgccagtcaacatgagaagtgaggcattcacctacaccagtgaacaccccgaaggattctataactggcaccacggagcggtgcagtatagtggaggtagatttaccatccctcgcggagtaggaggcagaggagacagcggtcgtccgatcatggataactccggtcgggttgtcgcgatagtcctcggtggcgctgatgaaggaacacgaactgccctttcggtcgtcacctggaatagtaaagggaagacaattaagacgaccccggaagggacagaagagtggtccgcagcaccactggtcacggcaatgtgtttgctcggaaatgtgagcttcccatgcgaccgcccgcccacatgctatacccgcgaaccttccagagccctcgacatccttgaagagaacgtgaaccatgaggcctacgataccctgctcaatgccatattgcggtgcggatcgtctggcagaagcaaaagaagcgtcattgacgactttaccctgaccagcccctacttgggcacatgctcgtactgccaccatactgtaccgtgcttcagccctgttaagatcgagcaggtctgggacgaagcggacgataacaccatacgcatacagacttccgcccagtttggatacgaccaaagcggagcagcaagcgcaaacaagtaccgctacatgtcgcttaagcaggatcacaccgttaaagaaggcaccatggatgacatcaagattagcacctcaggaccgtgtagaaggcttagctacaaaggatactttctcctcgcaaaatgccctccaggggacagcgtaacggttagcatagtgagtagcaactcagcaacgtcatgtacactggcccgcaagataaaaccaaaattcgtgggacgggaaaaatatgatctacctcccgttcacggtaaaaaaattccttgcacagtgtacgaccgtctgaaagaaacaactgcaggctacatcactatgcacaggccgagaccgcacgcttatacatcctacctggaagaatcatcagggaaagtttacgcaaagccgccatctgggaagaacattacgtatgagtgcaagtgcggcgactacaagaccggaaccgtttcgacccgcaccgaaatcactggttgcaccgccatcaagcagtgcgtcgcctataagagcgaccaaacgaagtgggtcttcaactcaccggacttgatcagacatgacgaccacacggcccaagggaaattgcatttgcctttcaagttgatcccgagtacctgcatggtccctgttgcccacgcgccgaatgtaatacatggctttaaacacatcagcctccaattagatacagaccacttgacattgctcaccaccaggagactaggggcaaacccggaaccaaccactgaatggatcgtcggaaagacggtcagaaacttcaccgtcgaccgagatggcctggaatacatatggggaaatcatgagccagtgagggtctatgcccaagagtcagcaccaggagaccctcacggatggccacacgaaatagtacagcattactaccatcgccatcctgtgtacaccatcttagccgtcgcatcagctaccgtggcgatgatgattggcgtaactgttgcagtgttatgtgcctgtaaagcgcgccgtgagtgcctgacgccatacgccctggccccaaacgccgtaatcccaacttcgctggcactcttgtgctgcgttaggtcggccaatgctgaaacgttcaccgagaccatgagttacttgtggtcgaacagtcagccgttcttctgggtccagttgtgcatacctttggccgctttcatcgttctaatgcgctgctgctcctgctgcctgccttttttagtggttgccggcgcctacctggcgaaggtagacgcctacgaacatgcgaccactgttccaaatgtgccacagataccgtataaggcacttgttgaaagggcagggtatgccccgctcaatttggagatcactgtcatgtcctcggaggttttgccttccaccaaccaagagtacattacctgcaaattcaccactgtggtcccctccccaaaaatcaaatgctgcggctccttggaatgtcagccggccgctcatgcagactatacctgcaaggtcttcggaggggtctacccctttatgtggggaggagcgcaatgtttttgcgacagtgagaacagccagatgagtgaggcgtacgtcgaattgtcagcagattgcgcgtctgaccacgcgcaggcgattaaggtgcacactgccgcgatgaaagtaggactgcgtattgtgtacgggaacactaccagtttcctagatgtgtacgtgaacggagtcacaccaggaacgtctaaagacttgaaagtcatagctggaccaatttcagcatcgtttacgccattcgatcataaggtcgttatccatcgcggcctggtgtacaactatgacttcccggaatatggagcgatgaaaccaggagcgtttggagacattcaagctacctccttgactagcaaggatctcatcgccagcacagacattaggctactcaagccttccgccaagaacgtgcatgtcccgtacacgcaggcctcatcaggatttgagatgtggaaaaacaactcaggccgcccactgcaggaaaccgcacctttcgggtgtaagattgcagtaaatccgctccgagcggtggactgttcatacgggaacattcccatttctattgacatcccgaacgctgcctttatcaggacatcagatgcaccactggtctcaacagtcaaatgtgaagtcagtgagtgcacttattcagcagacttcggcgggatggccaccctgcagtatgtatccgaccgcgaaggtcaatgccccgtacattcgcattcgagcacagcaactctccaagagtcgacagtacatgtcctggagaaaggagcggtgacagtacactttagcaccgcgagtccacaggcgaactttatcgtatcgctgtgtgggaagaagacaacatgcaatgcagaatgtaaaccaccagctgaccatatcgtgagcaccccgcacaaaaatgaccaagaatttcaagccgccatctcaaaaacatcatggagttggctgtttgcccttttcggcggcgcctcgtcgctattaattataggacttatgatttttgcttgcagcatgatgctgactagcacacgaagatgaccgctacgccccaatgatccgaccagcaaaactcgatgtacttccgaggaactgatgtgcataatgcatcaggctggtacattagatccccgcttaccgcgggcaatatagcaacactaaaaactcgatgtacttccgaggaagcgcagtgcataatgctgcgcagtgttgccacataaccactatattaaccatttatctagcggacgccaaaaactcaatgtatttctgaggaagcgtggtgcataatgccacgcagcgtctgcataacttttattatttcttttattaatcaacaaaattttgtttttaacatttc

SEQ ID NO: 16 - protein, 64 aa, Sindbis virus
SAAPLVTAMCLLGNVSFPCDRPPTCYTREPSRALDILEENVNHEAYDTLLNAILRCGSSGRSKR

SEQ ID NO: 17 - protein, 423 aa, Sindbis virus
SVIDDFTLTSPYLGTCSYCHHTVPCFSPVKIEQVWDEADDNTIRIQTSAQFGYDQSGAASANKYRYMSLKQDHTVKEGTMDDIKISTSGPCRRLSYKGYFLLAKCPPGDSVTVSIVSSNSATSCTLARKIKPKFVGREKYDLPPVHGKKIPCTVYDRLKETTAGYITMHRPRPHAYTSYLEESSGKVYAKPPSGKNITYECKCGDYKTGTVSTRTEITGCTAIKQCVAYKSDQTKWVFNSPDLIRHDDHTAQGKLHLPFKLIPSTCMVPVAHAPNVIHGFKHISLQLDTDHLTLLTTRRLGANPEPTTEWIVGKTVRNFTVDRDGLEYIWGNHEPVRVYAQESAPGDPHGWPHEIVQHYYHRHPVYTILAVASATVAMMIGVTVAVLCACKARRECLTPYALAPNAVIPTSLALLCCVRSANA

SEQ ID NO: 18 - DNA, 51 bp, Artificial sequence
  Linker
  CDS (1)..(51)
agatctggtggcggtggctcgggcggtggtgggtcgggtggcggcggatct

SEQ ID NO: 19 - protein, 17 aa, Artificial sequence
  Linker
RSGGGGSGGGGSGGGGS

SEQ ID NO: 20 - DNA, 45 bp, Artificial sequence
  Linker
  CDS (1)..(45)
ggtggcggtggctcgggcggtggtgggtcgggtggcggcggatct

SEQ ID NO: 21 - protein, 15 aa, Artificial sequence
  Linker
GGGGSGGGGSGGGGS

SEQ ID NO: 22 - DNA, 1050 bp, Bluetongue virus 10
  CDS (1)..(1050): VP7 gene
atggacactatcgccgcaagagcactcactgtgatgcgagcatgtgctacgcttcaagaggcaagaattgtgttggaagccaatgtgatggaaattttggggatagctatcaataggtacaatggactcactttacgaggagtgacgatgcgcccgacctcgttagcacaaagaaatgagatgttttttatgtgtttggatatgatgctgtctgctgctgggataaatgttggaccgatatcgccagactatactcaacatatggctacgattggtgtactagcaacaccggaaataccttttacaacggaagcggcgaatgaaatagcacgagtgactggggagacttcgacatgggggccagcgcgtcagccttatggtttcttccttgaaactgaggaaaccttccaaccagggaggtggttcatgcgcgccgctcaagcagtaactgcagtagtgtgcggtccggatatgattcaagtgtcacttaatgctggagcgagaggagatgtacaacagatatttcagggtcgtaatgatcccatgatgatatatttagtgtggaggagaatcgaaaactttgcgatggcgcaaggtaattcacagcaaactcaagcgggtgtgactgtcagtgttggtggagttgacatgagggcgggacgcattatagcgtgggatggacaggccgcgctgcatgtgcataatccgacacaacagaatgcgatggtgcaaatacaggttgtgttctatatatctatggataaaactttaaaccagtaccccgctttgactgctgagattttcaatgtttacagcttcagggaccacacatggcatgggctaagaacggcgatattaaacagaaccacactgccaaacatgctgccaccaatcttcccaccaaatgatcgagatagcatcttaactcttctacttttatctacacttgctgatgtttacactgttttaaggccagagtttgcgattcacggcgtaaatccgatgccagggccgctcacacgtgctattgcgcgcgccgcctatgtgtag

SEQ ID NO: 23 - protein, 349 aa, Bluetongue virus 10
MDTIAARALTVMRACATLQEARIVLEANVMEILGIAINRYNGLTLRGVTMRPTSLAQRNEMFFMCLDMMLSAAGINVGPISPDYTQHMATIGVLATPEIPFTTEAANEIARVTGETSTWGPARQPYGFFLETEETFQPGRWFMRAAQAVTAVVCGPDMIQVSLNAGARGDVQQIFQGRNDPMMIYLVWRRIENFAMAQGNSQQTQAGVTVSVGGVDMRAGRIIAWDGQAALHVHNPTQQNAMVQIQVVFYISMDKTLNQYPALTAEIFNVYSFRDHTWHGLRTAILNRTTLPNMLPPIFPPNDRDSILTLLLLSTLADVYTVLRPEFAIHGVNPMPGPLTRAIARAAYV

SEQ ID NO: 24 - DNA, 585 bp, Coccidioides immitis
  CDS (1)..(585): Ag2/PRA gene
atgcagttctctcacgctctcatcgctctcgtcgctgccggcctcgccagtgcccagctcccagacatcccaccttgcgctctcaactgcttcgttgaggctctcggcaacgatggctgcactcgcttgaccgacttcaagtgccactgctccaagcctgagctaccaggacagatcactccttgcgttgaggaggcctgccctctcgacgcccgtatctccgtctccaacatcgtcgttgaccagtgctccaaggccggtgtcccaattgacatcccaccagttgacaccaccgccgctcccgagccatccgagaccgctgagcccaccgctgagccaaccgaggagcccactgccgagcctaccgctgagcccaccgctgagccgactcatgagcccaccgaggagcccactgccgtcccaaccggcactggcggtggtgtccccactggcaccggttccttcaccgtcactggcagaccaactgcctccaccccagctgagttcccaggtgctggctccaacgtccgtgccagcgttggcggcattgctgctgctctcctcggtctcgctgcctacctgtaa

SEQ ID NO: 25 - protein, 194 aa, Coccidioides immitis
MQFSHALIALVAAGLASAQLPDIPPCALNCFVEALGNDGCTRLTDFKCHCSKPELPGQITPCVEEACPLDARISVSNIVVDQCSKAGVPIDIPPVDTTAAPEPSETAEPTAEPTEEPTAEPTAEPTAEPTHEPTEEPTAVPTGTGGGVPTGTGSFTVTGRPTASTPAEFPGAGSNVRASVGGIAAALLGLAAYL

SEQ ID NO: 26 - DNA, 906 bp, Streptococcus pneumoniae
  CDS (1)..(906): PspA gene
gaagaatctcccgtagccagtcagtctaaagctgagaaagactatgatgcagcgaagaaagatgctaagaatgcgaaaaaagcagtagaagatgctcaaaaggctttagatgatgcaaaagctgctcagaaaaaatatgacgaggatcagaagaaaactgaggagaaagccgcgctagaaaaagcagcgtctgaagagatggataaggcagtggcagcagttcaacaagcgtatctagcctatcaacaagctacagacaaagccgcaaaagacgcagcagataagatgatagatgaagctaagaaacgcgaagaagaggcaaaaactaaatttaatactgttcgagcaatggtagttcctgagccagagcagttggctgagactaagaaaaaatcagaagaagctaaacaaaaagcaccagaacttactaaaaaactagaagaagctaaagcaaaattagaagaggctgagaaaaaagctactgaagccaaacaaaaagtggatgctgaagaagtcgctcctcaagctaaaatcgctgaattggaaaatcaagttcatagactagaacaagagctcaaagagattgatgagtctgaatcagaagattatgctaaagaaggtttccgtgctcctcttcaatctaaattggatgccaaaaaagctaaactatcaaaacttgaagagttaagtgataagattgatgagttagacgctgaaattgcaaaacttgaagatcaacttaaagctgctgaagaaaacaataatgtagaagactactttaaagaaggtttagagaaaactattgctgctaaaaaagctgaattagaaaaaactgaagctgaccttaagaaagcagttaatgagccagaaaaaccagctccagctccagaaactccagccccagaa

SEQ ID NO: 27 - protein, 302 aa, Streptococcus pneumoniae
EESPVASQSKAEKDYDAAKKDAKNAKKAVEDAQKALDDAKAAQKKYDEDQKKTEEKAALEKAASEEMDKAVAAVQQAYLAYQQATDKAAKDAADKMIDEAKKREEEAKTKFNTVRAMVVPEPEQLAETKKKSEEAKQKAPELTKKLEEAKAKLEEAEKKATEAKQKVDAEEVAPQAKIAELENQVHRLEQELKEIDESESEDYAKEGFRAPLQSKLDAKKAKLSKLEELSDKIDELDAEIAKLEDQLKAAEENNNVEDYFKEGLEKTIAAKKAELEKTEADLKKAVNEPEKPAPAPETPAPE

(19) United States
(12) Patent Application Publication
(10) Pub. No.: US 2007/0275873 A1
(43) Pub. Date: Nov. 29, 2007

(54) Methods and Compositions Comprising Protein L Immunoglobulin Binding Domains for Cell-Specific Targeting

(76) Inventors: Hans Walter Heidner, Boerne, TX (US); William Brown Klimstra, Shreveport, LA (US); Katherine Diana Ryman, Shreveport, LA (US)

Correspondence Address: MYERS BIGEL SIBLEY & SAJOVEC, PO BOX 37428, RALEIGH, NC, 27627, US

(21) Appl. No.: 10/593,841

(22) PCT Filed: Apr. 29, 2004
(86) PCT No.: PCT/US04/13281

§ 371 Date: May 17, 2007

(51) Int. Cl. A61K 48/00; C07H 21/04; A61K 39/395

(52) U.S. Cl. 514/2; 435/235.1; 435/243; 435/320.1; 435/69.7; 530/402; 536/23.4; 424/178.1

(57) ABSTRACT

The present invention provides a fusion protein comprising a first amino acid sequence of at least one immunoglobulin-binding domain of Protein L and a second amino acid sequence of a peptide or protein that does not bind an immunoglobulin Fc region, as well as methods of making and using the fusion protein of this invention.

### BACKGROUND OF THE INVENTION

1. Field of the Invention

The present invention is directed to the use of immunoglobulin binding domains of Protein L to target substances to specific cell types.

2. Background Art

Protein L (PpL) is a bacterial cell wall protein that is expressed by approximately 10% of Peptostreptococcus magnus isolates (36, 55). PpL is an immunoglobulin (Ig)-binding protein and its expression has been correlated with virulence (36, 55, 67). PpL is a multidomain protein that contains four or five (depending of bacterial strain) highly homologous, repeated extracellular Ig-binding domains (designated B1-B5), 72 to 76 amino acids in length (37,54). Individual B domains retain Ig binding activity and studies have shown that each B domain possesses two separate Ig-binding sites (designated site 1 and site 2) (24, 25, 31). The Ig-binding properties of PpL are distinctly different from those of protein A (derived from Staphylococcus aureus) (19) and protein G (derived from group C and G Streptococci) (11, 66), which predominantly bind to the Fc region of IgG. PpL binds to Ig light chains, and therefore, binds to all classes of Ig (1, 10). Specifically, PpL binds with high affinity to the framework region of the variable domain (V_{L}) of kappa (subgroups κI, κIII, and κIV) light chains (18, 58), and binding does not interfere with the antigen-binding site of the Ig (1). PpL binds Igs from a broad range of mammalian species, and displays particularly high affinity for Ig of human, mouse, rat and swine origin (15). As Ig binding by PpL is not dependent on the class or antigen-binding properties of the Ig, and because the majority of human Igs contain kappa light chains (57, 82), PpL is able to bind 50% or more of the polyclonal antibodies in human serum (25, 57). PpL binds at least 40% of the antibodies present in mouse serum (57). Protein L is available commercially for the purification and detection of antibodies.

A major goal in vaccine technology is to deliver an antigen directly to cells of the immune system in a subject to elicit an effective immune response. Primary targets for such directed delivery are dendritic cells (DC). DC express receptors specific for the Fc region of immunoglobulin (Ig) G (FcγR), and of several other classes of Ig, and therefore, DCs can capture microbes/antigens that are bound by Ig (immune complexes [ICs]) (3, 26, 65). IC binding to FcγRs can lead to internalization of the IC/FcγR complex, DC activation and maturation (65). Acquisition of exogenous antigens by this FcR-mediated pathway can result in presentation of antigen-derived peptides in the context of MHC-I, a process that has been termed cross-presentation, which plays an important role in initiating CTL responses.

Gene therapy protocols also rely on targeted expression of therapeutic genes in specific cell types. Thus, the gene therapy vector must comprise a targeting molecule on the surface, which directs the vector to specific cell types in which expression of a therapeutic nucleic acid would be beneficial, and not to cells that could be harmed by introduction of the vector and/or its therapeutic nucleic acid The present invention addresses these issues in the art of targeted cell delivery by providing compositions for cell-specific targeting that comprise one or more binding domains of Protein L, which binds the light chain region of immunoglobulin molecules. Further provided are methods of making these compositions and using them in therapeutic protocols.

### BRIEF DESCRIPTION OF THE DRAWINGS

FIGS. 1A-B show the structure of PpL/E2 fusion proteins. A. Sindbis viruses were constructed that contain 1, 2, 3 or 4 PpL Ig binding domains attached at N-terminal extensions of the E2 glycoprotein. All viruses except that designated L1LN contain the PpL sequences attached to E2 through an intervening linker peptide, the structure of which is shown at the top. Virus L1LN contains a single PpL Ig-binding domain fused directly to E2. B. Three alternative sequences of PpL Ig-binding domain #1 have been constructed and fused to E2. The sequence of the wild type (L1), the non-glycosylated variant (ND/SK) and the Ig-binding negative (IBN) versions of the domain are shown. The underlined sequence (NGS) in L1 represents the single N-linked glycosylation signal present in the domain. This signal is ablated by changing the N residue to D and S residue to K as shown. The underlined residues identified as 1 and 2 in the IBN sequence identify the mutations that ablate the Ig-binding activity of sites 1 and 2, respectively.

FIG. 2 is a schematic for targeting a vaccine antigen to Fc receptor-bearing cells by coupling to Ig-binding domain(s) of Protein L. 1) The fusion protein consisting of Protein L and the antigen of interest is synthesized in cell culture or expressed in vivo using a vaccine vector. 2) Ig is bound to Protein L via the variable region of the kappa light chain. 3) Fc region of captured Ig will bind to the Fc receptor on target cell and then the entire Ig/protein complex will be internalized. In some cells (e.g., dendritic cells), the internalized antigen will be processed and then presented to Th cells in the context of MHC Class II and/or to Tc cells in the context of MHC class 1 proteins.

FIG. 3 is a schematic for targeting a vaccine vector to Fc receptor-bearing cells by coupling to Ig-binding domain(s) of Protein L. 1) Vaccine vector with Protein L-containing fusion proteins on its surface. 2) Ig bound to Protein L via variable region of kappa light chain. 3) The host provides this Ig in the body and it does not matter what the antigen specificity of the Ig is as long as it has a kappa light chain. 4) Fc region of captured It will bind to the Fc receptor on Fc receptor-positive cell and then the entire Ig/vector complex will be internalized. Internalization can lead to infection of the cell and expression of the antigen. In most cells, antigen-derived peptides will be presented to Tc cells in the context of MHC class I proteins.

FIG. 4 is a schematic for targeting a gene therapy vector to a cell type of interest using Ig-binding domain(s) of Protein L. 1) Gene therapy vector with Protein L-containing fusion proteins on its surface. 2) Gene of interest to be inserted into chromosome of target cell. 3) Ig Fab₂ fragment bound to gene therapy vector via capture by Protein L fusion protein. The Ig used in this type of targeting can be a monoclonal antibody chosen based on its ability to bind to a surface marker present on the target cell. 4) The target cell expresses a surface marker that is bound by the Fab fragment of the captured Fab₂. 5) Fab region of captured Ig Fab₂ fragment will bind to the surface receptor on the target cell and then the entire Fab₂/vector complex will be internalized.

### SUMMARY OF THE INVENTION

The present invention provides a fusion protein comprising, consisting of, or consisting essentially of, a first amino acid sequence of at least one immunoglobulin-binding domain of Protein L and a second amino acid sequence of a peptide or protein that does not bind an immunoglobulin Fc region. In various embodiments, the second amino acid sequence can be a protein or peptide, a protein of a virus, and/or a protein of a vector comprising a nucleic acid encoding an immunogenic or therapeutic protein or peptide.

In another embodiment, the present invention provides a composition comprising, consisting of, or consisting essentially of, the fusion protein of this invention, complexed with an immunoglobulin molecule or a Fab₂ fragment of an immunoglobulin molecule.

Further provided are nucleic acids encoding a fusion protein of this invention and vectors and virus particles comprising a nucleic acid encoding a fusion protein of this invention. Also provided are cells comprising each of these compositions.

In additional embodiments, the present invention provides a method of making a fusion protein comprising a first amino acid sequence of at least one immunoglobulin-binding domain of Protein L and a second amino acid sequence of a peptide or protein that does not bind an immunoglobulin Fc region, comprising: a) culturing cells comprising a recombinant nucleic acid encoding a fusion protein comprising a first amino acid sequence of at least one immunoglobulin-binding domain of Protein L and a second amino acid sequence of a peptide or protein that does not bind an immunoglobulin Fc region under conditions whereby the recombinant nucleic acid is expressed to produce the fusion protein; and b) collecting the fusion protein from the cells.

In addition, the present invention provides a method of delivering a fusion protein and/or a composition of this invention to an Fc receptor-bearing cell of a subject comprising, consisting of, or consisting essentially of, administering to the subject an effective amount of the fusion protein and/or composition.

Furthermore, the present invention provides a method of delivering a therapeutic or immunogenic protein or peptide to an Fc-bearing receptor cell in a subject, comprising, consisting of, or consisting essentially of, administering to the subject an effective amount of a fusion protein of this invention.

The present invention additionally provides a method of eliciting an immune response in a subject, comprising administering to the subject an effective amount of a composition comprising, consisting of, or consisting essentially of: a) a fusion protein comprising a first amino acid sequence of at least one immunoglobulin-binding domain of Protein L and a second amino acid sequence of an immunogenic protein or peptide; and b) an Fab₂ fragment of an immunoglobulin molecule specific for a receptor on the surface of the target cell or an immunoglobulin molecule capable of binding an Fc receptor on a cell.

Furthermore, the present invention provides a method of eliciting an immune response in a subject, comprising administering to the subject an effective amount of a composition comprising, consisting of, or consisting essentially of: a) a fusion protein comprising a first amino acid sequence of at least one immunoglobulin-binding domain of Protein L and a second amino acid sequence which is an amino acid sequence of a vector comprising a nucleic acid encoding an immunogenic protein or peptide; and b) an Fab₂ fragment of an immunoglobulin molecule specific for a receptor on the surface of the target cell or an immunoglobulin molecule capable of binding an Fc receptor on a cell.

In additional embodiments, the present invention provides a method of delivering a therapeutic substance to a target cell in a subject, comprising administering to the subject an effective amount of a composition comprising: a) a fusion protein comprising a first amino acid sequence of at least one immunoglobulin-binding domain of Protein L and a second amino acid sequence of a therapeutic protein or peptide; and b) a Fab₂ fragment of an antibody specific for a receptor on the surface of the target cell.

A method of delivering a therapeutic substance to a target cell in a subject is also provided, comprising administering to the subject an effective amount of a composition comprising: a) a fusion protein comprising a first amino acid sequence of at least one immunoglobulin-binding domain of Protein L and a second amino acid sequence which is an amino acid sequence of a vector comprising a nucleic acid encoding an immunogenic or therapeutic protein or peptide; and b) a Fab₂ fragment of an antibody specific for a receptor on the surface of the target cell.

The present invention also provides a method of treating cancer in a subject in need thereof, comprising administering to the subject an effective amount of a composition comprising: a) a fusion protein comprising a first amino acid sequence of at least one immunoglobulin binding domain of Protein L and a second amino acid sequence of a substance that is toxic to the cancer cell; and b) a Fab₂ fragment of an antibody specific for a receptor on the surface of a cancer cell of the subject.

Additionally provided herein is a method of treating cancer in a subject in need thereof, comprising administering to the subject an effective amount of a composition comprising: a) a fusion protein comprising a first amino acid sequence of at least one immunoglobulin binding domain of Protein L and a second amino acid sequence which is an amino acid sequence of a vector comprising a nucleic acid encoding a substance that is toxic to the cancer cell; and b) a Fab₂ fragment of an antibody specific for a receptor on the surface of a cancer cell of the subject.

Also provided herein is a method of treating cancer in a subject in need thereof, comprising administering to the subject an effective amount of a composition comprising: a) a fusion protein comprising a first amino acid sequence of at least one immunoglobulin binding domain of Protein L and a second amino acid sequence which is an amino acid sequence of an oncolytic virus; and b) an Fab₂ fragment of an antibody specific for a receptor on the surface of a cancer cell of the subject.

Various other objectives and advantages of the present invention will become apparent from the following detailed description.

### DETAILED DESCRIPTION OF THE INVENTION

As used herein, “a,” “an” or “the” can mean one or more than one. For example, “a” cell can mean a single cell or a multiplicity of cells.

The present invention is based on the unexpected discovery that a variety of compounds, such as antigens and vectors, can be targeted to specific cell types by exploiting the ability of Protein L (from Peptostreptococcus magnus) to bind the kappa light chain of immunoglobulin (Ig) molecules without interfering with the antigen binding site or Fc binding site of the Ig protein.

Protein L comprises four or five (depending on strain) highly homologous, repeated domains, each of which has the ability to bind immunoglobulin (Ig) molecules that contain kappa light chains (the majority of human and mouse Igs have kappa light chains) at the framework region of the variable light chain domain. When an Ig is bound to Protein L, the Fc region of the Ig is free to bind Fc receptors, which are present on various immune cells, and the Fab region of the Ig is available to interact with its specific antigen.

The nucleic acid and amino acid sequence of each of the Ig binding domains of Protein L of this invention are known (Kastern et al. Infect. Immun. 58:1217-1222 (1990) (Ref. 36); Kastern et al. J. Biol. Chem. 267:12820-12825 (1992) (Ref. 37); Murphy et al. Molec. Microbiol. 12:911-920 (1994) (Ref. 54); Murphy et al. “Nucleotide sequence of the gene for peptostreptococcal protein L” DNA Seq. 4:259-265 (1994); Genbank Accession No. M86697; Genbank Accession No. L04466; U.S. Pat. No. 6,162,903, the entire contents of each of which are incorporated by reference herein for their teachings of Protein L characterization and sequence) and are provided in the attached Sequence Listing. Thus, the fusion proteins of this invention can be constructed from these sequences as well as sequences that are functionally equivalent to these known sequences (see e.g., NDSK and IBN variants in Examples). A functionally equivalent amino acid sequence is an amino acid sequence that can have substitutions, deletions and/or additions to the known amino acid sequence that do not impart a change in the Ig binding activity of the Ig binding domain of Protein L. Any substitution, deletion and/or addition would be readily introduced into these known sequences by one of ordinary skill in the art using routine procedures and such altered amino acid sequences would be tested according to routine protocols to identify those amino acid sequences that retain the Ig binding activity of the unaltered amino acid sequence. Furthermore, a nucleic acid sequence encoding any such altered amino acid sequence would be readily identified by one of ordinary skill and would include any combination of nucleotides that encode the amino acid sequence of this invention. Due to the degeneracy of the genetic code, it would be readily understood that a large number of different nucleic acid sequences could encode the same amino acid sequence and all such nucleic acid sequences are included within the scope of this invention.

Thus, the present invention employs the binding affinity of Protein L for Ig molecules to target proteins and vectors to specific cell types, such as cells of the immune system and/or cells involved in gene therapy and other therapeutic protocols. Specifically, a fusion protein is produced according to the methods described herein, comprising one or more Ig-binding domains of Protein L fused with a protein or peptide that can be an antigen or therapeutic substance itself or a component of a virus (e.g., an oncolytic virus), a viral vector (e.g., adenovirus, retrovirus, AAV, alphavirus, vaccinia virus, etc.) or other vector that carries either free protein(s)/peptide(s), or a nucleic acid encoding immunogenic and/or otherwise therapeutic peptide(s) or polypeptide(s).

For applications wherein the target cell is an Fc receptor-bearing cell (e.g., M cell, dendritic cell, macrophage, mast cell, B lymphocytes, NK cells, neutrophils, monocytes, etc.) the Protein L fusion protein (consisting of free antigen, vector-expressed antigen and/or vector) is bound (either in vitro or in vivo) to kappa light chain-containing Igs with a functional Fc region (the antigen specificity is irrelevant) to form a fusion protein/Ig. The complex binds, via the Fc region of the Ig of the complex, Fc receptors on Fc receptor-bearing cells, which internalizes the ligand/receptor complex, thereby delivering the antigen or vector to the target cell.

With respect to their ability to target an antigen to cells of the immune system (e.g., dendritic cells, B lymphocytes, macrophages) the protein L Ig-binding domains can also function as an adjuvant, significantly enhancing the immune responses mounted against the antigens to which they are fused. In addition, targeting antigens to dendritic cells using this strategy can result in significant cross-presentation of antigen-derived peptides. Briefly, dendritic cells that acquire exogenous antigens via the FcR-mediated pathway are able to present peptides derived from the captured antigens in the contexts of both MHC-I and MHC-II. Because protein L also binds Ig from some domesticated animals (e.g., canines), and some animals of agricultural importance (e.g., swine) this vaccine technology is applicable to both humans and selected animal species.

The Protein L fusion protein of this inventor can also be used to target proteins, peptides, toxins, compounds and/or vectors to cells (e.g. tumor cells, stem cells, neurons, etc.) that express specific surface proteins (e.g., cancer antigens, specific CD antigens) via interaction between the Fab regions of the bound Ig and a specific molecule on the cell surface. For this application, the protein L fusion protein can be bound to Fab fragments (i.e., Ig fragments lacking an Fc region), instead of intact Ig so that Fc-mediated interactions would not interfere with Fab-mediated interactions. In this embodiment, the antigen-specificity of the antibody, the specific cell type, and the specific molecular target, are known. The Ig of the fusion protein/Ig complex will interact via the Fab₂ region with its specific antigen on a cell to form a complex that is internalized by the cell, facilitating delivery of a protein, peptide, toxin, compound, or vector to the cell.

Thus, the present invention provides a fusion protein comprising a first amino acid sequence of at least one immunoglobulin-binding domain of Protein L and a second amino acid sequence of a peptide or protein that does not bind an immunoglobulin Fc region. In some embodiments, the fusion protein of this invention can comprise one, two, three, four, five, six, seven, eight, nine, or ten Ig binding domains of Protein L and these binding domains can be in any order and/or combination and/or frequency in the same fusion protein (e.g., all can be different binding domains; all can be the same binding domain; some can be the some and others different; some can be from one species and others from another species, etc.). Furthermore, the fusion protein of this invention can comprise one or more Ig binding domains of Protein L at any position relative to the second amino acid sequence (e.g., at the amino terminus, at the carboxy terminus, within the second amino acid sequence and/or any combination thereof).

Thus, in various embodiments, the fusion protein of this invention comprises two immunoglobulin binding domains of Protein L, three immunoglobulin binding domains of Protein L, four immunoglobulin binding domains of Protein L, and/or five immunoglobulin binding domains of Protein L.

The proviso that the second amino acid sequence of the fusion protein of this invention is a peptide or protein that does not bind an immunoglobulin Fc region means that the second amino acid sequence of a fusion protein of this invention does not comprise a peptide, protein or binding domain that is known to bind to the Fc region of the heavy chain of an Ig molecule. Examples of such proteins that comprise domains that bind the Fc region of Ig molecules include Protein A, Protein G, Protein H and Protein M.

As used herein, the term “fusion protein” means a polypeptide, protein or peptide comprising a first amino acid sequence that is connected, linked or joined to a second amino acid sequence and wherein the first and second amino acid sequences are not connected, linked or joined in the same way in nature.

Also as used herein, the terms “peptide,” “protein” and “polypeptide” are used to describe a chain of amino acids, which correspond to those encoded by a nucleic acid. A peptide usually describes a chain of amino acids of from two to about 30 amino acids and polypeptide or protein usually describes a chain of amino acids having more than about 30 amino acids. The term polypeptide or protein can refer to a linear chain of amino acids or it can refer to a chain of amino acids that have been processed and folded into a functional protein. As presented herein, the terms protein and polypeptide can be used interchangeably. It is understood, however, that 30 is an arbitrary number with regard to distinguishing peptides and polypeptides and the terms can be used interchangeably for a chain of amino acids around 30.

The peptides and polypeptides of the present invention are obtained by isolation and purification of the peptides and polypeptides from cells where they are produced naturally or by expression of a recombinant and/or synthetic nucleic acid encoding the peptide or polypeptide. The peptides and polypeptides of this invention can be obtained by chemical synthesis, by proteolytic cleavage of a polypeptide and/or by synthesis from nucleic acid encoding the peptide or polypeptide.

It is also understood that the peptides and polypeptides of this invention can contain conservative substitutions where a naturally occurring amino acid is replaced by one having similar properties and which does not alter the function of the polypeptide. Such conservative substitutions are well known in the art. Thus, it is understood that, where desired, modifications and changes, which are distinct from the substitutions which enhance immunogenicity, can be made in the nucleic acid and/or amino acid sequence of the peptides and polypeptides of the present invention and still obtain a peptide or polypeptide having like or otherwise desirable characteristics. Such changes can occur in natural isolates or can be synthetically introduced using site-specific mutagenesis, the procedures for which, such as mis-match polymerase chain reaction (PCR), are well known in the art. One of skill in the art will also understand that polypeptides and nucleic acids that contain modified amino acids and nucleotides, respectively (e.g., to increase the half-life and/or the therapeutic efficacy of the molecule), can be used in the methods of the invention.

In certain embodiments, the fusion protein of this invention can comprise a linker sequence, which can be present between the first amino acid sequence and the second amino acid sequence and/or between each binding domain of a fusion protein comprising multiple binding domains. Thus, in some embodiments, the fusion protein of this invention can comprise a first amino acid sequence jointed to a second amino acid sequence by a linker amino acid sequence. A desirable linker amino acid sequence of this invention is an amino acid sequence that does not have ordered secondary structure and does not interfere with domain folding. Such amino acid sequences would be readily identified and tested by one of skill in the art according to routine protocols, such as those described in the Examples section herein. Thus, a fusion protein of this invention can comprise one linker sequence or more than one linker sequence. In other embodiments, the fusion protein of this invention does not contain a linker sequence. Thus, the first amino acid sequence and the second amino acid sequence of the fusion protein of this invention can be immediately adjacent to one another on the fusion protein and/or separated by a linker sequence of amino acids. The linker sequence can be 1, 2, 3, 4, 5, 6, 7, 8, 9, 10, 11, 12, 13, 14, 15, 16, 17, 18, 19, 20, 21, 22, 23, 24, 25, 50, 60, 70, 80, 90, 100, 125, 150, 175, 200, or greater than 200 amino acids in length and can comprise any amino acids.

Furthermore, a fusion protein of this invention can comprise more than one first amino acid sequence and/or more than second amino acid sequence. Thus, the first amino acid sequence and the second amino acid sequence can be present in the fusion protein in a ratio of 100:1, 90:1, 80:1, 70:1, 60;1, 50:1, 40:1, 30:1. 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50, 1:60, 1:70, 1:80, 1:90 and/or 1:100 or more.

In some embodiments, the linker sequence comprises at least three amino acids, at least five amino acids, at least ten amino acids, at least 15 amino acids, at least 20 amino acids, at least 25 amino acids and/or at least 30 amino acids. Also as noted above, a fusion protein of this invention can comprise more than one linker amino acid, which can be present in any combination and/or order (e.g., a fusion protein comprising a first linker sequence of at least five amino acids and a second linker sequence of at least 15 amino acids).

In a specific embodiment, the linker amino acid sequence of this invention can comprise, consist of or consist essentially of the amino acid sequence (Gly-Gly-Gly-Gly-Ser)₃ (33) (SEQ ID NO:21). In other embodiments the linker amino acid sequence of this invention can comprise, consist of or consist essentially of RSGGGGSGGGGSGGGGS (SEQ ID NO:19)

Also provided in the present invention is a composition comprising a fusion protein of this invention bound to an Ig molecule or a fragment of an Ig molecule to form a fusion protein/Ig complex. A fragment of an Ig molecule of this invention can include, but is not limited to Fab, Fab₂. An Ig or Ig fragment of this invention can also be “humanized” or otherwise genetically engineered to contain portions derived from different host species (e.g., an Ige that contains an Fab region from a mouse Ig and an Fc region of a human Ig), as described herein and according to procedures well known in the art. An Ig fragment of this invention can be produced by methods well known in the art. A complex of the fusion protein and the Ig or Ig fragment is formed by binding of the Protein L Ig binding domain(s) present in the fusion protein with the kappa light chain of the Ig molecule(s) and/or Ig fragment(s). A complex of this invention can comprise a fusion protein bound to Igs only, Ig fragments only or a combination of both.

Any type of immunoglobulin molecule (i.e., antibody) can be used in the present invention. The term “antibodies” as used herein refers to all types of immunoglobulins, including IgG, IgM, IgA, IgD, and IgE. The antibodies can be monoclonal or polyclonal and can be of any species of origin, including, e.g., mouse, rat, rabbit, horse, or human. (Walker et al., Molec. Immunol. 26, 403-11 (1989)). Antibody fragments that retain specific binding to the protein or epitope of this invention are included within the scope of the term “antibody” and include, for example, Fab, Fab₂, F(ab′)₂, and Fc fragments, and the corresponding fragments obtained from antibodies other than IgG. Such fragments can be produced by known techniques. The antibodies can be chimeric or humanized, particularly when they are used for therapeutic purposes Monoclonal antibodies of the present invention can be prepared using any technique that provides for the production of antibody molecules by continuous cell lines in culture. These include, but are not limited to, the hybridoma technique, the human B-cell hybridoma technique, and the EBV-hybridoma technique (Kohler et al. (1975) Nature 256:495-497; Kozbor et al. (1985) J. Immunol. Methods 81:31-42; Cote et al. (1983) Proc. Natl. Acad Sci. 80:2026-2030; Cole et al. (1984) Mol. Cell Biol. 62:109-120). Briefly, the procedure is as follows: an animal is immunized with antigen or immunogenic fragments or conjugates thereof. For example, haptenic oligopeptides of antigen can be conjugated to a carrier protein to be used as an immunogen. Lymphoid cells (e.g., splenic lymphocytes) are then obtained from the immunized animal and fused with immortalizing cells (e.g., myeloma or heteromyeloma) to produce hybrid cells. The hybrid cells are screened to identify those that produce the desired antibody.

Human hybridomas that secrete human antibody can be produced by the Kohler and Milstein technique. Hybridoma production in rodents, especially mouse, is a very well established procedure and thus, stable murine hybridomas provide an unlimited source of antibody of select characteristics. As an alternative to human antibodies, the mouse antibodies can be converted to chimeric murine/human antibodies by genetic engineering techniques. (Oi et al., Bio Techniques 4(4):214-221 (1986); Sun et al., Hybridoma 5 (1986)).

In addition, techniques developed for the production of “chimeric antibodies,” i.e., the splicing of mouse antibody genes to human antibody genes to obtain a molecule with appropriate antigen specificity and biological activity, can be used (Morrison, et al. Proc. Natl. Acad. Sci. 81:6851-6855 (1984); Neuberger et al. Nature 312:604-608 (1984); Takeda et al. Nature 314:452-454 (1985)). Alternatively, techniques described for the production of single chain antibodies can be adapted, using methods known in the art, to produce antigen-specific single chain antibodies. Antibodies with related specificity, but of distinct idiotypic composition, can be generated by chain shuffling from random combinatorial immunoglobulin libraries (Burton, Proc. Natl. Acad. Sci. 88:11120-3 (1991)).

Antibodies can also be produced by inducing in vivo production in the lymphocyte population or by screening immunoglobulin libraries or panels of highly specific binding reagents as disclosed in the literature (Orlandi et al. Proc. Natl. Acad. Sci. 86:3833-3837 (1989); Winter et al. Nature 349:293-299 (1991)).

Polyclonal antibodies used to carry out the present invention can be produced by immunizing a suitable animal (e.g., rabbit, goat, etc.) with an antigen, collecting immune serum from the animal, and separating the polyclonal antibodies from the immune serum, in accordance with known procedures. Depending on the host species, various adjuvants can be used to increase immunological response. Such adjuvants include, but are not limited to, Freund's, mineral gels such as aluminum hydroxide, and surface active substances such as lysolecithin, pluronic polyols, polyanions, peptides, oil emulsions, keyhole limpet hemocyanin, and dinitrophenol. Among adjuvants used in humans, BCG (bacilli Calmette-Guerin) and Corynebacterium parvum are especially preferable.

Monoclonal antibodies of this invention can be used to produce anti-idiotypic (paratope-specific) antibodies. (See e.g., McNamara et al. Science 220:1325-26 (1984); Kennedy et al. Science 232:220 (1986)). These antibodies resemble the epitope and thus can be used as an antigen to stimulate an immune response against the antigen, or to screen other antibodies for the ability to specifically bind to an epitope of interest.

The fusion protein/Ig complex of this invention can be formed in vitro, ex vivo or in vivo. For in vivo complex formation, only the fusion protein of this invention can be administered to a subject or the fusion protein and an Ig molecule can be administered to the subject, either simultaneously or in sequence. Ig molecules present within the subject have kappa light chains to which the fusion protein binds at the Protein L binding domain. The Ig molecules that bind the fusion protein within the subject can be produced naturally by the subject and/or introduced into the subject.

For example, in an embodiment wherein the fusion protein of this invention is delivering a toxic substance to a cancer cell, Ig molecules specific for a cancer antigen on the surface of the subject's cancer cells can be bound to he fusion protein ex vivo and the complex can be administered to the subject. Alternatively, the fusion protein can be administered to the subject and the Ig molecules specific for the cancer antigen can be present in the subject and/or can also be administered to the subject (either concurrently and/or before or after administration of the fusion protein). In the latter embodiment, the fusion protein will form a complex with the cancer antigen-specific Ig molecules within the subject. In either embodiment, the fusion protein/Ig molecule complex will bind to the surface of a cancer cell bearing the cancer antigen and the fusion protein carrying the toxic substance will be delivered to the cancer cell.

The route of administration to the subject can be any route that results in contact between the fusion protein/Ig complex and the target cell. Thus for example, intravenous administration is suitable for target cells in the hepatic, splenic, renal cardiac and circulatory or hematopoietic systems. The complex and/or fusion protein and/or Ig can also be administered by catheterization of the artery or vein leading to the target organ, thereby allowing the localized administration of the complex. The complex and/or fusion protein and/or Ig can also be administered by inspiration when the target cells are in the respiratory system.

The present invention further provides a fusion protein of this invention, wherein the second amino acid sequence is an amino acid sequence of a virus protein. In this embodiment, the presence of the Ig binding domain in a protein of a virus particle allows for the binding of Ig with a virus particle. The Ig/virus particle complex is then targeted to a specific cell type (i.e., a cell bearing an Fc receptor on the surface that binds the Fc region of the Ig of the Ig/virus particle complex or a cell bearing a molecule that binds the Fab region of the Ig of the Ig/virus particle complex). The fusion protein/Ig complex is internalized by the target cell and nucleic acid present in the virus particle is delivered to and expressed within the cell.

The virus of this invention can be any virus that is suitable for introduction into a subject as a virus particle to impart a therapeutic effect. The virus particle can itself be a vaccine antigen and/or the virus particle of this invention can be a vaccine vector and/or a gene therapy vector. In this first embodiment, the virus particle can be a virus particle that does not cause disease in a subject because it has been attenuated by any of a variety of well known methods. In the latter two embodiments, the virus particle can be a recombinant virus particle that has been engineered according to well known methods in the art to be a virus particle comprising a nucleic acid encoding an immunogenic and/or otherwise therapeutic protein or molecule. Such a virus particle is capable of complexing with an Ig binding domain of Protein L for delivery to a target cell but is not capable of generating infectious virus particles within the target cell.

Specifically, the particle is internalized by the target cell via binding of the Ig/virus particle complex to the surface of the target cell and the nucleic acid carried by the virus particle is expressed within the target cell but the virus particle is deficient in nucleic acid encoding all of the virus proteins required for production of new virus particles. In this manner, the virus particle delivers a therapeutic nucleic acid to the target cell but does not produce infectious virus particles that could infect the subject.

Thus, an additional embodiment of this invention is a virus particle comprising a fusion protein of this invention. The virus particle of this invention can be of any virus suitable for administration to a subject as a vaccine and/or as a vector. For example, the virus of this invention can be, but is not limited to, alphavirus, lentivirus, retrovirus, adenovirus, adeno-associated virus (AAV), flavivirus, herpesvirus, poxvirus, rhabdovirus, picomavirus, bacteriophage, plant virus and any other virus and/or viral replicon derived from such viruses now known or later identified to be suitable for administration to a subject as a vaccine and/or a vector. Also included among the viruses of this invention are chimeric or pseudotyped viruses, wherein viral proteins of at least two different viruses are present in the same virus particle (e.g., a lentivirus core surrounded by an envelope comprising VSV-G protein). The production and use of such chimeric and pseudotye viruses are well known in the art (see. e.g., 42, 43, 60, 72, 76, 80, 91, 93, the entire contents of each of which are incorporated herein for their teachings of chimeric and/or pseudotyped viruses). The nucleotide and amino acid sequences of the viruses of this invention are known in the art and are available in the literature.

As an example, in one embodiment of the present invention, the fusion protein of this invention can be a glycoprotein that can be, for example, an E2 glycoprotein of an alphavirus such as Sindbis virus and the present invention therefore includes an alphavirus particle comprising a fusion protein of this invention. The nucleotide and amino acid sequences of Sindbis virus glycoproteins are readily available in the literature and are provided in the Sequence Listing attached hereto (Heidner et al. J. Virol. 68:8064-8070 (1994) (Ref. 28); McKnight et al. J. Virol. 70:1981-1989 (1996) (Ref. 50); Rice et al. “Nucleotide sequence of the 26S mRNA of Sindbis virus and deduced sequence of the encoded virus structural proteins” PNAS USA 78:2062-2066 Genbank Accession No. M13818; Genbank Accession No. NC_{—}001547; the entire contents of each of which are incorporated by reference herein for their teachings of Sindbis virus and its sequences). The production and characterization of such virus particles comprising a fusion protein of this invention are described in detail in the Examples section herein.

In another embodiment, the present invention provides a fusion protein of this invention, wherein the second amino acid sequence is an amino acid sequence of a non-viral vector comprising a nucleic acid encoding an immunogenic or therapeutic protein or peptide. Examples of non-viral vectors of this invention include, but are not limited to, proteins incorporated into liposomes, iscomes, protein micelles, recombinant bacteria such as E. coli or other microbial agent, sequences of the PpL and fusion protein contained in nucleic acid (e.g., DNA) vaccines.

In a further embodiment of this invention, the fusion protein of this invention can comprise, as the second amino acid sequence, an amino acid sequence of an immunogenic and/or otherwise therapeutic protein or peptide. Thus, for example, in one embodiment, the fusion protein of this invention can comprise a second amino acid sequence that is an amino acid sequence of an immunogenic peptide. In this embodiment, the fusion protein can be administered to the subject either alone or as a complex with an Ig molecule of this invention (e.g., which can be an Fab₂ fragment). When the fusion protein is administered alone, it can form a complex with Ig molecules that are present in the subject naturally or with Ig molecules that have been introduced into the subject. The fusion protein or peptide is delivered to a target cell as part of an Ig complex, where it is taken in and functions directly as an immunogenic protein or peptide.

In an embodiment wherein the fusion protein comprises a second amino acid sequence that is a therapeutic protein or peptide, the fusion protein is complexed with Ig or an Ig fragment either in vivo or ex vivo and is delivered to a target cell where it is taken in and functions directly as a therapeutic protein or peptide.

In an embodiment wherein the nucleic acid within the virus particle or vector encodes an antigen and the goal is to deliver the Ig/virus particle complex to an Fc receptor-bearing immune cell, the specificity of the Fab region of the Ig of the complex is irrelevant.

In an embodiment wherein nucleic acid within the virus particle or vector encodes a therapeutic protein and the goal is to deliver the Ig/ virus particle complex to a specific cell bearing a known target surface molecule, the Fab region of the Ig of the complex must be specific for the target surface molecule.

The present invention also provides a nucleic acid encoding the fusion protein of this invention. This nucleic acid can be present as a free nucleic acid or it can be present within a vector sequence. The nucleic acid and/or a vector comprising the nucleic acid can be within any cell that can express the nucleic acid. In one embodiment, the nucleic acid, vector and cell can be used to produce the fusion proteins of this invention ex vivo for administration to a subject as described herein. In another embodiment, the nucleic acid of this invention and vectors comprising the nucleic acid of this invention can be administered to the subject for production of a fusion protein in vivo. The fusion protein produced in vivo from this nucleic acid can form a complex with antibodies that are naturally present within a subject and/or the fusion protein can form a complex with antibodies that are administered to the subject. The resulting fusion protein/Ig complex is then bound by a target cell within a subject and the fusion protein is taken inside the target cell, where it imparts its immunogenic and/or therapeutic effect.

“Nucleic acid” as used herein refers to single- or double-stranded molecules which can be DNA, comprised of the nucleotide bases A, T, C and G, or RNA, comprised of the bases A, U (substitutes for T), C, and G. The nucleic acid can represent a coding strand or its complement. Nucleic acids can be identical in sequence to the sequence that is naturally occurring, or they can include alternative codons, which encode the same amino acid as that found in the naturally occurring sequence. Furthermore, nucleic acids can include codons that represent conservative substitutions of amino acids, as are well known in the art. The nucleic acids of this invention can also comprise any nucleotide analogs and/or derivatives as are well known in the art.

As used herein, the term “isolated nucleic acid” means a nucleic acid separated or substantially free from at least some of the other components of the naturally occurring organism, for example, the cell structural components commonly found associated with nucleic acids in a cellular environment and/or other nucleic acids. The isolation of nucleic acids can therefore be accomplished by well-known techniques such as cell lysis followed by phenol plus chloroform extraction, followed by ethanol precipitation of the nucleic acids. The nucleic acids of this invention can be isolated from cells according to methods well known in the art for isolating nucleic acids. Alternatively, the nucleic acids of the present invention can be synthesized according to standard protocols well described in the literature for synthesizing nucleic acids. Modifications to the nucleic acids of the invention are also contemplated, provided that the essential structure and function of the peptide or polypeptide encoded by the nucleic acid is maintained.

The nucleic acid of this invention can be part of a recombinant nucleic acid construct comprising any combination of restriction sites and/or functional elements as are well known in the art that facilitate molecular cloning and other recombinant DNA manipulations. Thus, the present invention further provides a recombinant nucleic acid construct comprising a nucleic acid encoding a peptide and/or polypeptide of this invention.

The present invention further provides a vector comprising a nucleic acid encoding a peptide and/or polypeptide of this invention. The vector can be an expression vector which contains all of the genetic components required for expression of the nucleic acid in cells into which the vector has been introduced, as are well known in the art. The expression vector can be a commercial expression vector or it can be constructed in the laboratory according to standard molecular biology protocols. The expression vector can comprise, for example, viral nucleic acid including, but not limited to, vaccinia virus, adenovirus, retrovirus, alphavirus and/or adeno-associated virus nucleic acid. The nucleic acid or vector of this invention can also be in a liposome or a delivery vehicle, which can be taken up by a cell via receptor-mediated or other type of endocytosis.

The nucleic acid of this invention can be in a cell that is a cell expressing the nucleic acid whereby a peptide and/or polypeptide of this invention is produced in the cell. In addition, the vector of this invention can be in a cell that is a cell expressing the nucleic acid of the vector whereby a peptide and/or polypeptide of this invention is produced in the cell. It is also contemplated that the nucleic acids and/or vectors of this invention can be present in a host (e.g., a bacterial cell, a cell line, a transgenic animal, etc.) that can express nucleic acids encoding the peptides and/or polypeptides of the present invention.

In some embodiments, the present invention provides a method of making a fusion protein comprising a first amino acid sequence of at least one immunoglobulin-binding domain of Protein L and a second amino acid sequence of a peptide or protein that does not bind an immunoglobulin Fc region, comprising: a) culturing cells comprising a recombinant nucleic acid encoding a fusion protein comprising a first amino acid sequence of at least one immunoglobulin-binding domain of Protein L and a second amino acid sequence of a peptide or protein that does not bind an immunoglobulin Fc region under conditions whereby the recombinant nucleic acid is expressed to produce the fusion protein; and b) collecting the fusion protein from the cells.

For production of the fusion proteins of this invention in prokaryotes, there are numerous E. coli (Escherichia coli) expression vectors known to one of ordinary skill in the art useful for the expression of nucleic acid encoding proteins such as fusion or chimeric proteins. Other microbial hosts suitable for use include bacilli, such as Bacillus subtilis, and other enterobacteria, such as Salmonella, Serratia, as well as various Pseudomonas species. These prokaryotic hosts can support expression vectors that will typically contain sequences compatible with the host cell (e.g., an origin of replication). In addition, any number of a variety of well-known promoters will be present, such as the lactose promoter system, a tryptophan (Trp) promoter system, a B-lactamase promoter system, or a promoter system from phage lambda. The promoters will typically control expression, optionally with an operator sequence and have ribosome binding site sequences for example, for initiating and completing transcription and translation. If necessary, an amino terminal methionine can be provided by insertion of a Met codon 5′ and in-frame with the coding sequence of the protein. Also, the carboxy-terminal extension of the protein can be removed using standard oligonucleotide mutagenesis procedures.

Additionally, yeast expression systems and baculovirus systems, which are well known in the art, can be used to produce the fusion peptides and polypeptides of this invention.

The vectors of this invention can be transferred into a cell by well-known methods, which vary depending on the type of cell host. For example, calcium chloride transfection is commonly utilized for prokaryotic cells, whereas calcium phosphate treatment, lipofection or electroporation can be used for other cell hosts.

The nucleic acid of this invention can be any nucleic acid that functionally encodes the fusion proteins, peptides and/or polypeptides of this invention. To functionally encode the fusion proteins, peptides and polypeptides (i.e., allow the nucleic acids to be expressed), the nucleic acid of this invention can include, for example, antibiotic resistance markers, origins of replication and/or expression control sequences, such as, for example, a promoter (constitutive or inducible), an enhancer and necessary information processing sites, such as ribosome binding sites, RNA splice sites, polyadenylation sites and transcriptional terminator sequences.

Examples of expression control sequences useful in this invention include promoters derived from metallothionine genes, actin genes, immunoglobulin genes, CMV, SV40, adenovirus, bovine papilloma virus, etc. A nucleic acid encoding a selected peptide or polypeptide can readily be determined based upon the genetic code for the amino acid sequence of the selected peptide or polypeptide and many nucleic acids will encode any selected peptide or polypeptide. Modifications in the nucleic acid sequence encoding the peptide or polypeptide are also contemplated. Modifications that can be useful are modifications to the sequences controlling expression of the peptide or polypeptide to make production of the peptide or polypeptide inducible or repressible as controlled by the appropriate inducer or repressor. Such methods are standard in the art. The nucleic acid of this invention can be generated by means standard in the art, such as by recombinant nucleic acid techniques and by synthetic nucleic acid synthesis or in vitro enzymatic synthesis.

It is also an aspect of this invention that the various compositions described above can be used in methods for delivering a fusion protein of this invention to a target cell within a subject. Thus, in a further embodiment, the present invention provides a method of delivering a fusion protein of this invention to an Fc receptor-bearing cell of a subject comprising administering to the subject an effective amount of the fusion protein.

Also provided is method of delivering a fusion protein of this invention to an Fc-receptor bearing cell of a subject, comprising administering to the subject an effective amount of a composition of this invention comprising a fusion protein/Ig complex. The fusion protein can be targeted to an Fc receptor-bearing cell or a cell bearing a molecule on its surface that is bound by the Ig of the fusion protein/Ig complex.

In a further embodiment, a method of delivering a therapeutic or immunogenic protein or peptide to an Fc-bearing receptor cell in a subject is provided, comprising administering to the subject an effective amount of a fusion protein of this invention wherein the second amino acid sequence is an amino acid sequence of a vector comprising a nucleic acid encoding an immunogenic or therapeutic protein or peptide.

Additionally provided is a method of delivering a therapeutic substance to a target cell in a subject, comprising administering to the subject an effective amount of a composition comprising: a) a fusion protein comprising a first amino acid sequence of at least one immunoglobulin-binding domain of Protein L and a second amino acid sequence of a therapeutic protein or peptide; and b) an Fab₂ fragment of an antibody specific for a protein (e.g., receptor) on the surface of the target cell.

The present invention further provides a method of delivering a therapeutic substance to a target cell in a subject, comprising administering to the subject an effective amount of a composition comprising: a) a fusion protein comprising a first amino acid sequence of at least one immunoglobulin-binding domain of Protein L and a second amino acid sequence which is an amino acid sequence of a vector comprising a nucleic acid encoding an immunogenic or therapeutic protein or peptide; and b) an Fab₂ fragment of an antibody specific for a protein on the surface of the target cell.

Another embodiment of this invention includes a method of eliciting an immune response in a subject, comprising administering to the subject an effective amount of a composition comprising: a) a fusion protein comprising a first amino acid sequence of at least one immunoglobulin-binding domain of Protein L and a second amino acid sequence of an immunogenic protein or peptide; and b) an Fab₂ fragment of an immunoglobulin molecule specific for a receptor on the surface of the target cell or an immunoglobulin molecule capable of binding an Fc receptor.

Also provided herein is a method of eliciting an immune response in a subject, comprising administering to the subject an effective amount of a composition comprising: a) a fusion protein comprising a first amino acid sequence of at least one immunoglobulin-binding domain of Protein L and a second amino acid sequence which is an amino acid sequence of a vector comprising a nucleic acid encoding an immunogenic protein or peptide; and b) an Fab₂ fragment of an immunoglobulin molecule specific for a receptor on the surface of the target cell or an immunoglobulin molecule capable of binding an Fc receptor.

In other embodiments, the present invention provides a method of treating cancer in a subject in need thereof, comprising administering to the subject an effective amount of a composition comprising: a) a fusion protein comprising a first amino acid sequence of at least one immunoglobulin binding domain of Protein L and a second amino acid sequence of a substance that is toxic to or otherwise detrimental to the vitality (e.g., a chemotherapeutic agent or drug) the cancer cell; and b) an Fab₂ fragment of an antibody specific for a protein on the surface of a cancer cell of the subject. Examples of toxic substances of this invention include, but are not limited to, toxins and radioisotopes. As one example, the vector that is delivered to the target cell can also be designed to express a “suicide protein” such as the thymidine kinase protein of herpes simplex virus. The subject is then given a prodrug such as acyclovir, which will only be activated (converted to acyclovir-Triphosphate) in the target cell. Only the target cell dies, since acyclovir-Triphosphate is toxic to the cell.

Also provided herein is a method of treating cancer in a subject in need thereof, comprising administering to the subject an effective amount of a composition comprising: a) a fusion protein comprising a first amino acid sequence of at least one immunoglobulin binding domain of Protein L and a second amino acid sequence which is an amino acid sequence of a vector comprising a nucleic acid encoding a substance that is toxic to or otherwise detrimental to the vitality of the cancer cell; and b) an Fab₂ fragment of an antibody specific for a receptor on the surface of a cancer cell of the subject.

In an additional embodiment of this invention, a method is provided for treating cancer in a subject in need thereof, comprising administering to the subject an effective amount of a composition comprising: a) a fusion protein comprising a first amino acid sequence of at least one immunoglobulin binding domain of Protein L and a second amino acid sequence which is an amino acid sequence of an oncolytic virus; and b) an Fab₂ fragment of an immunoglobulin specific for a receptor on the surface of a cancer cell of the subject or an immunoglobulin that can bind an Fc receptor.

In some embodiments of the present invention, the toxic substance used to treat a cancer in a subject can be a genome of an oncolytic virus. Such viruses include, but are not limited to, alphaviruses (e.g., Sindbis virus), rhabdoviruses (e.g., VSV), (herpesviruses (e.g., herpes simplex), paramyxoviruses (e.g., Sendai virus), adenoviruses (e.g., adenovirus) and reoviruses (e.g., reovirus), as well as any other oncolytic virus now known or later identified. In these embodiments, as one example, an alphavirus particle comprising one or more Ig binding domains of Protein L in the E2 protein is bound to an Ig molecule that is specific for a protein on the surface of a cancer cell in a subject to form a complex and the complex is administered to the subject. The complex binds the cancer cell and is taken up by the cell, where the alphavirus proteins are produced that are toxic to the cancer cell.

In other embodiments, the fusion protein of this invention can be an alphavirus particle comprising one or more Ig binding domains of Protein L in the E2 protein, and wherein the alphavirus particle comprises an alphavirus genome-derived nucleic acid element that expresses a nucleic acid sequence that encodes a substance that is toxic to cancer cells. In this embodiment, the alphavirus protein is complexed with an Ig molecule that is specific for a protein on the surface of a cancer cell in a subject and complex is administered to the subject. The complex binds the cancer cell and is taken up by the cell, where the nucleic acid from the alphavirus particle is expressed to produce the toxic substance.

As used herein, an “effective amount” refers to an amount of a compound or composition that is sufficient to produce a desired effect, which can be a therapeutic or beneficial effect. The effective amount will vary with the age, general condition of the subject, the severity of the condition being treated, the particular biologically active agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation. (See, for example, Remington, The Science And Practice of Pharmacy (20th ed. 2000)).

Also as used herein, the terms “treat,” “treating” and “treatment” include any type of mechanism, action or activity that results in a change in the medical status of a subject, including an improvement in the condition of the subject (e.g., change or improvement in one or more symptoms and/or clinical parameters), delay in the progression of the condition, prevention or delay of the onset of a disease or illness, etc.

A therapeutic compound or substance of this invention is one that imparts a beneficial effect in a subject. Examples of such beneficial effects include, but are not limited to, treatment or prevention of an infection or disease, killing and/or arresting growth of tumor cells, restoration of a function in a cell comprising a defective protein by providing a functional replacement protein, etc.

An antigen of this invention can be a whole protein, a fragment of a protein, an immunogenic peptide, an antibody and/or T cell epitope and/or a T cell stimulatory peptide. Identification of immunogenic peptides, T cell stimulatory peptides, antibody and T cell epitopes and the like is carried out by methods well known in the art.

For example, an antigen of this invention can include, but is not limited to, influenza antigens, polio antigens, tetanus toxin and other tetanus antigens, herpes antigens [e.g., CMV, EBV, HSV, VZV (chicken pox virus)], mumps antigens, measles antigens, rubella antigens, diphtheria toxin or other diphtheria antigens, pertussis antigens, hepatitis (e.g., hepatitis A, hepatitis B, hepatitis C) antigens, smallpox antigens and adenovirus antigens.

An antigen of this invention can also include, but is not limited to, cancer antigens, infectious agent antigens, allergic reaction antigens (allergens), transplantation antigens, autoantigens and the like as are known in the art.

A cancer antigen (i.e., an antigen specifically associated with cancer cells) of this invention can include, for example, HER2/neu and BRCA1 antigens for breast cancer, MART-1/MelanA, gp100, tyrosinase, TRP-1, TRP-2, NY-ESO-1, CDK-4, β-catenin, MUM-1, Caspase-8, KIAA0205, HPVE7, SART-1, PRAME, and p15 antigens, members of the MAGE family, the BAGE family (such as BAGE-1), the DAGE/PRAME family (such as DAGE-1), the GAGE family, the RAGE family (such as RAGE-1), the SMAGE family, NAG, TAG-72, CA125, mutated proto-oncogenes such as p21ras, mutated tumor suppressor genes such as p53, tumor associated viral antigens (e.g., HPV16 E7), the SSX family, HOM-MEL-55, NY-COL-2, HOM-HD-397, HOM-RCC-1.14, HOM-HD-21, HOM-NSCLC-11, HOM-MEL-2.4, HOM-TES-11, RCC-3.1.3, NY-ESO-1, and the SCP family. Members of the MAGE family include, but are not limited to, MAGE-1, MAGE-2, MAGE-3, MAGE-4 and MAGE-11. Members of the GAGE family include, but are not limited to, GAGE-1, GAGE-6. See, e.g., review by Van den Eynde and van der Bruggen (1997) in Curr. Opin. Immunol. 9: 684-693, Sahin et al. (1997) in Curr. Opin. Immunol. 9: 709-716, and Shawler et al. (1997), the entire contents of which are incorporated by reference herein for their teachings of cancer antigens.

The cancer antigen can also be, but is not limited to, human epithelial cell mucin (Muc-1; a 20 amino acid core repeat for Muc-1 glycoprotein, present on breast cancer cells and pancreatic cancer cells), MUC-2, MUC-3, MUC-18, the Ha-ras oncogene product, carcino-embryonic antigen (CEA), the raf oncogene product, CA-125, GD2, GD3, GM2, TF, sTn, gp75, EBV-LMP 1 & 2, HPV-F4, 6, 7, prostatic serum antigen (PSA), prostate-specific membrane antigen (PSMA), alpha-fetoprotein (AFP), CO17-1A, GA733, gp72, p53, the ras oncogene product, β-HCG, gp43, HSP-70, p17 mel, HSP-70, gp43, HMW, HOJ-1, melanoma gangliosides, TAG-72, mutated proto-oncogenes such as p21ras, mutated tumor suppressor genes such as p53, estrogen receptor, milk fat globulin, telomerases, nuclear matrix proteins, prostatic acid phosphatase, protein MZ2-E, polymorphic epithelial mucin (PEM), folate-binding-protein LK26, truncated epidermal growth factor receptor (EGFR), Thomsen-Friedenreich (T) antigen, GM-2 and GD-2 gangliosides, polymorphic epithelial mucin, folate-binding protein LK26, human chorionic gonadotropin (HCG), pancreatic oncofetal antigen, cancer antigens 15-3,19-9, 549, 195, squamous cell carcinoma antigen (SCCA), ovarian cancer antigen (OCA), pancreas cancer associated antigen (PaA), mutant K-ras proteins, mutant p53, and chimeric protein p210_{BCR-ABL} and tumor associated viral antigens (e.g., HPV16 E7).

Treatment of cancer according to the present invention can be by the delivery of nucleic acids encoding proteins which destroy or arrest growth of the target cell (for example, a ribosomal toxin), indirectly stimulate destruction of target cell by natural effector cells (for example, strong antigens to stimulate immune system) or convert a precursor substance to a toxic substance which destroys the target cell (for example, a prodrug-activating enzyme). Encoded proteins could also destroy bystander tumor cells (for example with secreted antitumor antibody-ribosomal toxin fusion protein), indirectly stimulate destruction of bystander tumor cells (for example cytokines to stimulate immune system or procoagulant proteins causing local vascular occlusion) or convert a precursor substance to a toxic substance that destroys bystander tumor cells (e.g. enzyme which activates prodrug to diffusible drug). Also included is the delivery of genes encoding antisense transcripts or ribozymes that interfere with expression of cellular genes critical for tumor persistence (e.g., against aberrant myc transcripts in Burkitt's lymphoma or against bcr-abl transcripts in chronic myeloid leukemia).

The cancer antigen of this invention can also be an antibody produced by a B cell tumor (e.g., B cell lymphoma; B cell leukemia; myeloma; hairy cell leukemia), a fragment of such an antibody, which contains an epitope of the idiotype of the antibody, a malignant B cell antigen receptor, a malignant B cell immunoglobulin idiotype, a variable region of an immunoglobulin, a hypervariable region or complementarity determining region (CDR) of a variable region of an immunoglobulin, a malignant T cell receptor (TCR), a variable region of a TCR and/or a hypervariable region of a TCR. In one embodiment, the cancer antigen of this invention can be a single chain antibody (scFv), comprising linked V_{H}, and V_{L} domains, which retains the conformation and specific binding activity of the native idiotype of the antibody.

The present invention is in no way limited to the cancer antigens listed herein. Other cancer antigens be identified, isolated and cloned by methods known in the art such as those disclosed in U.S. Pat. No. 4,514,506, the entire contents of which are incorporated by reference herein.

The cancer to be treated by the compositions and methods of this invention can be, but is not limited to, B cell lymphoma, T cell lymphoma, myeloma, leukemia, hematopoietic neoplasias, thymoma, lymphoma, sarcoma, lung cancer, liver cancer, non-Hodgkins lymphoma, Hodgkins lymphoma, uterine cancer, adenocarcinoma, breast cancer, pancreatic cancer, colon cancer, lung cancer, renal cancer, bladder cancer, liver cancer, prostate cancer, ovarian cancer, primary or metastatic melanoma, squamous cell carcinoma, basal cell carcinoma, brain cancer, angiosarcoma, hemangiosarcoma, head and neck carcinoma, thyroid carcinoma, soft tissue sarcoma, bone sarcoma, testicular cancer, uterine cancer, cervical cancer, gastrointestinal cancer, and any other cancer now known or later identified (see, e.g., Rosenberg (1996) Ann. Rev. Med. 47:481-491, the entire contents of which are incorporated by reference herein).

Infectious agent antigens of this invention can include, but are not limited to, antigenic peptides or proteins encoded by the genomes of Hepadnaviridae including hepatitis A, B, C, D, E, F, G, etc. (e.g., HBsAg, HBcAg, HBeAg); Flaviviridae including human hepatitis C virus (HCV), yellow fever virus and dengue viruses; Retroviridae including human immunodeficiency viruses (HIV) (e.g., gp120, gp160, gp41, an active (i.e., antigenic) fragment of gp120, an active (i.e., antigenic) fragment of gp160 and/or an active (i.e., antigenic) fragment of gp41) and human T lymphotropic viruses (HTLV1 and HTLV2); Herpesviridae including herpes simplex viruses (HSV-1 and HSV-2), Epstein Barr virus (EBV), cytomegalovirus, varicella-zoster virus (VZV), human herpes virus 6 (HHV-6) human herpes virus 8 (HHV-8), and herpes B virus; Papovaviridae including human papilloma viruses; Rhabdoviridae including rabies virus; Paramyxoviridae including respiratory syncytial virus; Reoviridae including rotaviruses; Bunyaviridae including hantaviruses; Filoviridae including Ebola virus; Adenoviridae; Parvoviridae including parvovirus B-19; Arenaviridae including Lassa virus; Orthomyxoviridae including influenza viruses (e.g., NP, HA antigen); Poxviridae including Orf virus, molluscum contageosum virus, smallpox virus and Monkey pox virus; Togaviridae including Venezuelan equine encephalitis virus; Coronaviridae including corona viruses such as the severe acute respiratory syndrome (SARS) virus; and Picornaviridae including polioviruses; rhinoviruses; orbiviruses; picodnaviruses; encephalomyocarditis virus (EMV); Parainfluenza viruses, adenoviruses, Coxsackieviruses, Echoviruses, Rubeola virus, Rubella virus, human papillomaviruses, Canine distemper virus, Canine contagious hepatitis virus, Feline calicivirus, Feline rhinotracheitis virus, TGE virus (swine), Foot and mouth disease virus, simian virus 5, human parainfluenza virus type 2, human metapneuomovirus, enteroviruses, and any other pathogenic virus now known or later identified (see, e.g., Fundamental Virology, Fields et al., Eds., 3^{rd} ed., Lippincott-Raven, New York, 1996, the entire contents of which are incorporated by reference herein for the teachings of pathogenic viruses).

The antigen of this invention can be an antigenic peptide or protein of a pathogenic microorganism, which can include but is not limited to, Rickettsia, Chlamydia, Mycobacteria, Clostridia, Corynebacteria, Mycoplasma, Ureaplasma, Legionella, Shigella, Salmonella, pathogenic Escherichia coli species, Bordatella, Neisseria, Treponema, Bacillus, Haemophilus, Moraxella, Vibrio, Staphylococcus spp., Streptococcus spp., Campylobacter spp., Borrelia spp., Leptospira spp., Erlichia spp., Klebsiella spp., Pseudomonas spp., Helicobacter spp., and any other pathogenic microorganism now known or later identified (see, e.g., Microbiology, Davis et al, Eds., 4^{th} ed., Lippincott, New York, 1990, the entire contents of which are incorporated herein by reference for the teachings of pathogenic microorganisms).

Specific examples of microorganisms from which the antigen of this invention can be obtained include, but are not limited to, Helicobacter pylori, Chlamydia pneumoniae, Chlamydia trachomatis, Ureaplasma urealyticum, Mycoplasma pneumoniae, Staphylococcus aureus, Streptococcus pyogenes, Streptococcus pneumoniae, Streptococcus viridans, Enterococcus faecalis, Neisseria meningitidis, Neisseria gonorrhoeae, Treponema pallidum, Bacillus anthracis, Salmonella typhi, Vibrio cholera, Pasteurella pestis, Pseudomonas aeruginosa, Campylobacter jejuni, Clostridium difficile, Clostridium botulinum, Mycobacterium tuberculosis, Borrelia burgdorferi, Haemophilus ducreyi, Corynebacterium diphtheria, Bordetella pertussis, Bordetella parapertussis, Bordetella bronchiseptica, Haemophilus influenza, and enterotoxic Escherichia coli, as well as any microbe classified as a “select agent” by the Centers for Disease Control and Prevention (CDC), or as a “high consequence” livestock pathogen by the U.S. Department of Agriculture (USDA).

Antigens of this invention can be antigenic peptides or proteins from pathogenic protozoa, including, but not limited to, Plasmodium species (e.g., malaria antigens), Babeosis species, Schistosoma species, Trypanosoma species, Pneumocystis carnii, Toxoplasma species, Leishmania species, and any other protozoan pathogen now known or later identified.

Additionally, antigens of this invention can be antigenic peptides or proteins from pathogenic yeast and fungi, including, but not limited to, Aspergillus species, Candida species, Cryptococcus species, Histoplasma species, Coccidioides species, and any other pathogenic fungus now known or later identified.

Specific examples of various antigens of this invention include, but are not limited to, the influenza virus nucleoprotein (residues 218-226; Fu et al. (1997) J Virol. 71: 2715-2721), antigens from Sendai virus and lymphocytic choriomeningitis virus (An et al. (1997) J. Virol. 71: 2292-2302), the B1 protein of hepatitis C virus (Bruna-Romero et al. (1997) Hepatology 25: 470-477), gp 160 of HIV (Achour et al. (1996) J. Virol. 70: 6741-6750), amino acids 252-260 of the circumsporozoite protein of Plasmodium berghei (Allsopp et al. (1996) Eur. J. Immunol. 26: 1951-1958), the influenza A virus nucleoprotein (residues 366-374; Nomura et al. (1996) J. Immunol. Methods 193: 4149), the listeriolysin O protein of Listeria monocytogenes (residues 91-99; An et al. (1996) Infect. Immun. 64: 1685-1693), the E6 protein (residues 131-140; Gao et al. (1995) J. Immunol. 155: 5519-5526) and E7 protein (residues 21-28 and 48-55; Bauer et al. (1995) Scand. J. Immunol. 42: 317-323) of human papillomavirus type 16, the M2 protein of respiratory syncytial virus (residues 82-90 and 81-95; Hsu et al. (1995) Immunology 85: 347-350), the herpes simplex virus type 1 ribonucleotide reductase (Salvucci et al. (1995) J. Gen. Virol. 69: 1122-1131), the rotavirus VP7 protein (Franco et al. (1993) J. Gen. Virol. 74: 2579-2586), P. falciparum antigens (causing malaria) and hepatitis B surface antigen (Gilbert et al. (1997) Nature Biotech. 15: 1280-1283).

Transplantation antigens for use as an antigen of this invention include, but are not limited to, different antigenic specificities of HLA-A, B and C Class I proteins. Different antigenic specificities of HLA-DR, HLA-DQ, HLA-DP and HLA-DW Class II proteins can also be used (WHO Nomenclature Committee, Immunogenetics 16:135 (1992); Hensen et al., in Fundamental Immunology, Paul, Ed., pp. 577-628, Raven Press, New York, 1993; NIH Genbank and EMBL data bases).

The present invention also contemplates the use of allergic antigens or allergens, which can include, but are not limited to, environmental allergens such as dust mite allergens; plant allergens such as pollen, including ragweed pollen; insect allergens such as bee and ant venom; and animal allergens such as cat dander, dog dander and animal saliva allergens.

The present invention also provides autoantigens as an antigen of this invention, for example, to enhance self-tolerance to an autoantigen in a subject, such as an elderly person, in whom self-tolerance is impaired. Exemplary autoantigens of this invention can include, but are not limited to, myelin basic protein, islet cell antigens, insulin, collagen and human collagen glycoprotein 39, muscle acetylcholine receptor and its separate polypeptide chains and peptide epitopes, glutamic acid decarboxylase and muscle-specific receptor tyrosine kinase.

The nucleic acids of this invention that encode immunogenic and/or therapeutic proteins and/or molecules can include any nucleic acid that can be expressed in a eukaryotic system. Examples of nucleic acids that encode a therapeutic protein and that can be employed in the compositions and methods of this invention include, but are not limited to, glucocerebrosidase, adenosine deaminase, and blood coagulation factors such as factor VIII and factor IX. The nucleic acid of this invention can also encode a ribozyme or antisense sequence.

The fusion protein/Ig complexes of this invention can have a variety of specificities. In particular, there are a large number of cell surface molecules for which Ig molecules are already available. Examples of such molecules include, but are not limited to, the class I and class II major histocompatibility (MHC) antigens; receptors for cytokines and cell-type specific growth hormones, brain derived neurotrophic factor (BDNF), ciliary neurotrophic factor (CTNF), colony stimulating growth factors, endothelial growth factors, epidermal growth factors, fibroblast growth factors, glially derived neurotrophic factor, glial growth factors, gro-β/mip 2, hepatocyte growth factor, insulin-like growth factor, interferon (e.g., α-IFN, β-IFN, γ-IFN, consensus IFN, etc.), interleukin (e.g., L-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, Il-21, IL-22, IL-23, Il-24, Il-25, IL-26, IL-27, IL-28A, IL-28B, IL-29, etc.), keratinocyte growth factor, leukemia inhibitory factors, macrophage/monocyte chemotactic activating factor, nerve growth factor, neutrophil activating protein 2, platelet derived growth factor, stem cell factor, transforming growth factor, tumor necrosis factors and vascular endothial growth factor; cell adhesion molecules; transport molecules for metabolites such as amino acids; the antigen receptors of B- and T-lymphocytes; and/or receptors for lipoproteins.

The disease and/or disorder that can be treated by the methods of this invention can include any disease or disorder that can be treated by mounting an effective immune response to an antigen of this invention. For example, the methods of the present invention can be used to treat cancer, viral infections, bacterial infections, fungal infections, parasitic infections and/or other diseases and disorders that can be treated by eliciting an immune response in and/or delivering a therapeutic substance to cells of a subject of this invention.

There are many examples of bacterial and viral diseases that can be prevented and/or treated by the methods described herein. Specifically, the methods described herein can be used for the following infections and/or diseases: adenovirus, AIDS, antibiotic associated diarrhea, bacterial pneumonia, bovine herpes virus (BHV-1), chlamydia, croup, diphtheria, Clostridium difficile, cystitis, cytomegovirus (CMV), gastritis, gonorrhea, Helicobactor pylori, hepatitis A, hepatitis B, hepatitis C, herpes virus, HSV-1, HSV-2, human papilloma virus, influenza, Legionnaires disease, Lyme disease, malaria, multiple sclerosis, peptic ulcer, pertussis, psoriasis, rabies, respiratory syncytial virus (RSV), rheumatoid arthritis, rhinovirus, rotavirus, salmonella, strap throat, tetanus, travelers diarrhea, etc.

It is also contemplated that the compositions of this invention can be used as a vaccine or prophylactic composition and employed in methods of treating and/or preventing a disease or disorder in a subject, comprising administering to the subject an effective amount of the composition of this invention. In some embodiments, the vaccine can be administered to a subject who is identified to be at risk of contracting a particular disease or developing a particular disorder. Identification of a subject at risk can include, for example, evaluation of such factors as family history, genetic predisposition, age, environmental exposure, occupation, lifestyle and the like, as are well known in the art.

A subject of this invention can be any animal to which the compositions of this invention can be administered. In certain embodiments, the subject is a mammal (e.g., dog, cat, horse, goat, sheep, monkey, rabbit, pig, cow, guinea pig, hamster, gerbil, ferret, etc.) and in specific embodiments, the subject is a human.

In certain embodiments of this invention, a fusion protein of this invention or a nucleic acid encoding a fusion protein of this invention can be combined with an adjuvant (which can be either a polypeptide or a nucleic acid encoding a polypeptide).

Thus, the present invention further provides a composition comprising a fusion protein of this invention and an adjuvant and/or composition comprising an adjuvant in the form of a peptide or protein, as well as a nucleic acid encoding a fusion protein of this invention and a nucleic acid encoding an adjuvant. The adjuvant, in the form of a peptide or protein, can be a component of the fusion protein and/or a separate component of a composition comprising the fusion protein of this invention. The adjuvant in the form of a nucleic acid can be a component of the nucleic acid encoding the fusion protein and/or a separate component of the composition comprising the nucleic acid encoding the fusion protein of this invention. In a further embodiment, the adjuvant can be encoded by a nucleic acid sequence present in a vector of this invention. An adjuvant of this invention can be an amino acid sequence that is a peptide, a protein fragment or a whole protein that functions as the adjuvant, or the adjuvant can be a nucleic acid encoding a peptide, protein fragment or whole protein that functions as an adjuvant.

As used herein, “adjuvant” describes a substance that can be any immunomodulating substance capable of being combined with the fusion protein or nucleic acid of this invention to enhance, improve or otherwise modulate an immune response in a subject without deleterious effect on the subject.

An adjuvant of this invention can be, but is not limited to, for example, an immunostimulatory cytokine (including, but not limited to, GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules), SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Suitable adjuvants also include an aluminum salt such as aluminum hydroxide gel (alum), aluminum phosphate, or algannmulin, but can also be a salt of calcium, iron or zinc, or can be an insoluble suspension of acylated tyrosine, or acylated sugars, cationically or anionically derivatized polysaccharides, or polyphosphazenes.

Other adjuvants are well known in the art and include QS-21, Freund's adjuvant (complete and incomplete), aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion.

Additional adjuvants can include, for example, a combination of monophosphoryl lipid A, preferably 3-de-O-acylated monophosphoryl. lipid A (3D-MPL) together with an aluminum salt. An enhanced adjuvant system involves the combination of a monophosphoryl lipid A and a saponin derivative, particularly the combination of QS21 and 3D-MPL as disclosed in PCT publication number WO 94/00153 (the entire contents of which are incorporated herein by reference), or a less reactogenic composition where the QS21 is quenched with cholesterol as disclosed in PCT publication number WO 96/33739 (the entire contents of which are incorporated herein by reference). A particularly potent adjuvant formulation involving QS21 3D-MPL & tocopherol in an oil in water emulsion is described in PCT publication number WO 95/17210 (the entire contents of which are incorporated herein by reference). In addition, the nucleic acid of this invention can include an adjuvant by comprising a nucleotide sequence encoding a fusion protein of this invention and a nucleotide sequence that provides an adjuvant function, such as CpG sequences. Such CpG sequences, or motifs, are well known in the art.

An adjuvant of this invention, such as, for example, an immunostimulatory cytokine, can be administered before, concurrent with, and/or within a few hours, several hours, and/or 1, 2, 3, 4, 5, 6, 7, 8, 9, and/or 10 days before or after the administration of a composition of this invention to a subject.

Furthermore, any combination of adjuvants, such as immunostimulatory cytokines, can be co-administered to the subject before, after or concurrent with the administration of a composition of this invention. For example, combinations of immunostimulatory cytokines can consist of two or more immunostimulatory cytokines of this invention, such as GM/CSF, interleukin-2, interleukin-12, interferon-gamma, interleukin-4, tumor necrosis factor-α, interleukin-1, hematopoietic factor flt3L, CD40L, B7.1 co-stimulatory molecules and B7.2 co-stimulatory molecules. The effectiveness of an adjuvant or combination of adjuvants can be determined by measuring the immune response produced in response to administration of a composition of this invention to a subject with and without the adjuvant or combination of adjuvants, using standard procedures, as described herein and as known in the art.

Pharmaceutical compositions comprising a composition of this invention and a pharmaceutically acceptable carrier are also provided. The compositions described herein can be formulated for administration in a pharmaceutical carrier in accordance with known techniques. See, e.g., Remington, The Science And Practice of Pharmacy (latest edition). In the manufacture of a pharmaceutical composition according to embodiments of the present invention, the composition of this invention is typically admixed with a pharmaceutically acceptable carrier. By “pharmaceutically acceptable carrier” is meant a carrier that is compatible with other ingredients in the pharmaceutical composition and that is not harmful or deleterious to the subject. The carrier can be a solid or a liquid, or both, and is preferably formulated with the composition of this invention as a unit-dose formulation, for example, a tablet, which can contain from about 0.01 or 0.5% to about 95% or 99% by weight of the composition. The pharmaceutical compositions are prepared by any of the well-known techniques of pharmacy including, but not limited to, admixing the components, optionally including one or more accessory ingredients.

The pharmaceutical compositions of this invention include those suitable for oral, rectal, topical, inhalation (e.g., via an aerosol) buccal (e.g., sub-lingual), vaginal, parenteral (e.g., subcutaneous, intramuscular, intradermal, intraarticular, intrapleural, intraperitoneal, intracerebral, intraarterial, or intravenous), topical (i.e., both skin and mucosal surfaces, including airway surfaces) and transdermal administration, although the most suitable route in any given case will depend, as is well known in the art, on such factors as the species, age, gender and overall condition of the subject, the nature and severity of the condition being treated and/or on the nature of the particular composition (i.e., dosage, formulation) that is being administered.

Pharmaceutical compositions suitable for oral administration can be presented in discrete units, such as capsules, cachets, lozenges, or tables, each containing a predetermined amount of the composition of this invention; as a powder or granules; as a solution or a suspension in an aqueous or non-aqueous liquid; or as an oil-in-water or water-in-oil emulsion. Oral delivery can be performed by complexing a composition of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the composition and a suitable carrier (which can contain one or more accessory ingredients as noted above). In general, the pharmaceutical composition according to embodiments of the present invention are prepared by uniformly and intimately admixing the composition with a liquid or finely divided solid carrier, or both, and then, if necessary, shaping the resulting mixture. For example, a tablet can be prepared by compressing or molding a powder or granules containing the composition, optionally with one or more accessory ingredients. Compressed tablets are prepared by compressing, in a suitable machine, the composition in a free-flowing form, such as a powder or granules optionally mixed with a binder, lubricant, inert diluent, and/or surface active/dispersing agent(s). Molded tablets are made by molding, in a suitable machine, the powdered compound moistened with an inert liquid binder.

Pharmaceutical compositions suitable for buccal (sub-lingual) administration include lozenges comprising the composition of this invention in a flavored base, usually sucrose and acacia or tragacanth; and pastilles comprising the composition in an inert base such as gelatin and glycerin or sucrose and acacia.

Pharmaceutical compositions of this invention suitable for parenteral administration can comprise sterile aqueous and non-aqueous injection solutions of the composition of this invention, which preparations are preferably isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

The compositions can be presented in unit\dose or multi-dose containers, for example, in sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, an injectable, stable, sterile composition of this invention in a unit dosage form in a sealed container can be provided. The composition can be provided in the form of a lyophilizate, which can be reconstituted with a suitable pharmaceutically acceptable carrier to form a liquid composition suitable for injection into a subject. The unit dosage form can be from about 1 μg to about 10 grams, including any value in between these numbers, of the composition of this invention. When the composition is substantially water-insoluble, a sufficient amount of emulsifying agent, which is physiologically acceptable, can be included in sufficient quantity to emulsify the composition in an aqueous carrier. One such useful emulsifying agent is phosphatidyl choline.

Pharmaceutical compositions suitable for rectal administration are preferably presented as unit dose suppositories. These can be prepared by admixing the composition with one or more conventional solid carriers, such as for example, cocoa butter and then shaping the resulting mixture.

Pharmaceutical compositions of this invention suitable for topical application to the skin preferably take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical composition of the present invention with a lipophilic reagent (e.g., DMSO) that is capable of passing into the skin.

Pharmaceutical compositions suitable for transdermal administration can be in the form of discrete patches adapted to remain in intimate contact with the epidermis of the subject for a prolonged period of time. Compositions suitable for transdermal administration can also be delivered by iontophoresis (see, for example, Pharmaceutical Research 3:318 (1986)) and typically take the form of an optionally buffered aqueous solution of the composition of this invention. Suitable formulations can comprise citrate or bis\tris buffer (pH 6) or ethanol/water and can contain from 0.1 to 0.2M active ingredient.

An effective amount of a composition of this invention, the use of which is in the scope of present invention, will vary from composition to composition, and subject to subject, and will depend upon a variety of well known factors such as the age and condition of the patient and the form of the composition and route of delivery. An effective amount can be determined in accordance with routine pharmacological procedures known to those skilled in the art. As a general proposition, a dosage from about 0.1 μg/kg to about 50 mg/kg, including any value within this range (e.g., from about 1 μg/kg, 2 μg/kg, 3 μg/kg, 4 μg/kg, 5 μg/kg, 10 μg/kg, etc. to 1 mg/kg, 2 mg/kg, 3 mg/kg, 4 mg/kg, 5 mg/kg, 10 mg/kg, etc.), will be an effective amount, with all weights being calculated based upon the weight of the composition.

The frequency of administration of a composition of this invention can be as frequent as necessary to impart the desired therapeutic effect. For example, the composition can be administered one, two, three, four or more times per day, one, two, three, four or more times a week, one, two, three, four or more times a month, one, two, three or four times a year or as necessary to control the condition. In some embodiments, one, two, three or four doses over the lifetime of a subject can be adequate to achieve the desired therapeutic effect. The amount and frequency of administration of the composition of this invention will vary depending on the particular condition being treated or to be prevented and the desired therapeutic effect.

A noted above, the compositions of this invention can be administered to a cell of a subject either in vivo or ex vivo. For administration to a cell of the subject in vivo, as well as for administration to the subject, the compositions of this invention can be administered, for example, orally, parenterally (e.g., intravenously), by intramuscular injection, intradermally (e.g., by gene gun), by intraperitoneal injection, subcutaneous injection, transdermally, extracorporeally, topically or the like. Also, the composition of this invention can be pulsed onto dendritic cells, which are isolated or grown from a subject's cells, according to methods well known in the art, or onto bulk PBMC or various cell subfractions thereof from a subject.

If ex vivo methods are employed, cells or tissues can be removed and maintained outside the body according to standard protocols well known in the art while the compositions of this invention are introduced into the cells or tissues. For example, the nucleic acids and vectors of this invention can be introduced into cells via any gene transfer mechanism, such as, for example, virus-mediated gene delivery, calcium phosphate mediated gene delivery, electroporation, microinjection or proteoliposomes. The transduced cells can then be infused (e.g., in a pharmaceutically acceptable carrier) or transplanted back into the subject per standard methods for the cell or tissue type. Standard methods are known for transplantation or infusion of various cells into a subject.

Administration of the nucleic acids of this invention can be achieved by any one of numerous, well-known approaches, for example, but not limited to, direct transfer of the nucleic acids, in a plasmid or viral vector, or via transfer in cells or in combination with carriers such as cationic liposomes. Such methods are well known in the art and readily adaptable for use in the methods described herein.

As noted above, vectors employed in the methods of this invention can be any nucleotide construct used to deliver nucleic acid into cells, e.g., a plasmid or viral vector, such as alphaviral vectors (Pushko et al. Virology 239(2):389-401 (1997), retroviral vectors (Pastan et al. Proc. Natl. Acad Sci. U.S.A. 85:4486 (1988); Miller et al., Mol. Cell. Biol. 6:2895 (1986)), adenoviral vectors (Mitani et al. Hum. Gene Ther. 5:941-948, 1994), adeno-associated viral (AAV) vectors (Goodman et al., Blood 84:1492-1500, 1994), lentiviral vectors (Naldini et al., Science 272:263-267, 1996), pseudotyped retroviral vectors (Agrawal et al., Exper. Hematol. 24:738-747, 1996), and any other viral vector now known or later identified. Physical transduction techniques can also be used, such as liposome delivery and receptor-mediated and other endocytosis mechanisms (see, for example, Schwartzenberger et al., Blood 87:472-478, 1996). This invention can be used in conjunction with any of these or other commonly used nucleic acid transfer methods. Appropriate means for transfection, including viral vectors, chemical transfectants, or physico-mechanical methods such as electroporation and direct diffusion of DNA, are described by, for example, Wolff et al., Science 247:1465-1468 (1990) and Wolff. Nature 352:815-818 (1991).

The present invention is more particularly described in the following examples that are intended as illustrative only since numerous modifications and variations therein will be apparent to those skilled in the art.

### EXAMPLES

### Example 1

Alphaviruses are single-stranded, positive-sense RNA viruses, and are classified within the Togaviridae virus family (genus alphavirus) (84). The alphavirus genome can accommodate foreign gene sequences and many recombinant alphaviruses and alphavirus-based replicon vectors have been described (20, 64, 74, 75, 87). The most common in vivo application of alphavirus-based expression vectors is as recombinant vaccines (64, 75). Alphaviruses possess several properties that recommend them for use as vaccine vectors; including broad host range, availability of attenuated genotypes, ease of genetic manipulation, and high-level antigen expression. However, these properties alone do not ensure that vaccines based on recombinant alphaviruses will be capable of inducing effective adaptive immune responses against their expressed antigens. Effective stimulation of lymphocytes by vaccine antigens can be influenced by many factors, including the form of the antigen, the microenvironment of the antigen/lymphocyte interaction, and the participation of additional immune system components, such as dendritic cells (DCs). DCs function as important cellular components of innate immunity and efficiently detect and respond against infectious agents that enter the body through the skin or across mucosal surfaces (5, 26, 51). DCs also function as the primary antigen presenting cells of the adaptive immune system. Antigenic stimulation of immature DCs in the periphery results in DC maturation and migration to regional lymphoid tissues where they present processed antigen to T lymphocytes and participate in the generation of antigen-specific cellular and humoral immune responses (26, 65, 77). Because processing and presentation of antigens by DCs influences the magnitude, quality, and memory of the ensuing immune response, targeting vaccines to this important cell type is a rationale strategy for enhancing vaccine efficacy (5, 83).

Sindbis virus infects murine DCs in vivo (70, 71), and has been reported to display a limited tropism for human DCs grown in culture (21, 39). There is considerable interest in targeting alphavirus-based vaccines to DCs and strategies for enhancing viral tropism for DCs have been reported. Repeated passage of a laboratory strain of Sindbis virus on human monocyte-derived DC (MDDC) cultures generated a variant that displayed enhanced infectivity for DCs (21). This variant contained a single point mutation in the E2 glycoprotein (residue 160 E to G), and infected approximately 19% of the MDDCs when infections were performed at a multiplicity of infection (MOI) of 50 plaque forming units (pfu)/cell. By comparison, the consensus AR339 strain of Sindbis virus (TR339) (50), infected approximately 1% of MDDCs when virus was derived from a Chinese hamster ovary cell line, but could infect approximately 30% of MDDCs when the virus was derived from cultured mosquito cells (C6/36) (39). The enhanced infectivity of the mosquito-cell derived virus for MDDCs was attributed to the structures of the N-linked carbohydrate moieties on the viral glycoproteins, which are limited to high mannose forms (Man₃GlcNAc₂) when virus is grown in this cell type (32). High mannose oligosaccharides serve as ligands for the C-type lectins DC-SIGN and L-SIGN, which are expressed on defined populations of immature dendritic cells (6, 81), and on the MDDCs used in the study. Based on these results it was concluded that alphavirus-based vaccines could be more efficiently targeted to DC-SIGN-positive DCs by propagating the vectors under conditions that limit the processing of viral glycoprotein-linked carbohydrate groups to high mannose structures (39).

In the present invention, the construction and characterization of recombinant Sindbis viruses that express 1 to 4 PpL B domains as N-terminal extensions of the viral E2 glycoprotein are described. The recombinant viruses are shown to bind Ig in an antigen-independent manner and to recapitulate the species-specific Ig-binding characteristics of native PpL. The Ig-binding viruses displayed antibody-dependent enhancement (ADE) of infection, as virus incubation with Ig markedly enhanced the viral infectivity for a non-susceptible, FcγR-positive murine macrophage-like cell line. These phenotypes were directly linked to the Ig-binding property of the viruses as variants containing point mutations within the Ig-binding sites of PpL failed to bind Ig, and did not display ADE of infection of FcγR-positive cell types.

Cells and Growth Conditions.

BHK-21 cells were purchased from the American Type Culture Collection (ATCC) and were maintained in alpha minimum essential medium (MEM) supplemented with 10% donor calf serum (DCS), 10% tryptose phosphate broth (TPB) and antibiotics (MEM-complete). The murine monocyte/macrophage-derived cell line designated J774A.1 was purchased from the ATCC. J774A.1 cells were maintained in Dulbecco's modified Eagle's medium supplemented with 10% DCS and antibiotics (DMEM-complete).

Construction of Recombinant Viruses.

The parental virus used in this study is designated TRSB-E2S1 (28), and all recombinant viruses were ultimately constructed in the genetic background of this virus. TRSB-E2S1 contains two mutations (Gln for Arg at nsP3 residue 528 and Val for Ala at E1 residue 72) relative to the sequence of the consensus AR339 virus (50), neither of which is associated with any phenotype in cell culture or in vivo (40, 41). Nucleotide sequences encoding a Bgl II restriction site upstream of a 15 amino acid linker segment ([Gly₄Ser]₃) (33) were inserted into the cDNA clone of TRSB-E2S1 (pTRSB-E2S1) between the E2 and E3 genes using an overlapping PCR cloning strategy (85, 87). The resulting plasmid was designated pTRSB-E2S1-linker. Sequences encoding 1 to 4 Ig binding domains of protein L (derived from Peptostreptococcus magnus strain 312), were amplified by PCR using the pHDB1-4 plasmid as template (Affitech AS, Norway), and fused in frame and downstream of the E3 coding sequences using the same overlapping PCR technique. The oligonucleotides used in these reactions generated a Bgl II restriction site immediately downstream of the protein L sequences and amplified a product that extended upstream of the unique Aat II restriction site present within the capsid gene sequences. The amplicons were then digested with Aat II and Bgl II and inserted into a pTRSB-E2S1-linker from which a corresponding fragment had been removed. The resulting constructs were designated pTRSB-E2S1-L1, pTRSB-E2S1-L2, pTRSB-E2S1-L3, and pTRSB-E2S1-L4, and viruses derived from these constructs were designated L1, L2, L3, and L4, respectively. The first Ig binding domain of protein L encodes a potential site for N-linked glycosylation (Asn-Gly-Ser) at residues 31-33 (numbering according to (25)). Ig binding domains 2-4 encode Asp-Gly-Lys at the comparable positions (37). In order to eliminate the potential for N-linked glycosylation at this site during virus growth in vertebrate cells, the Asn-Gly-Ser sequence in Ig binding domain 1 was mutated to Asp-Gly-Lys using a PCR-based mutagenesis procedure. The resulting construct was designated pTRSB-E2S1-ND/SK, and virus derived from this plasmid was designed ND/SK. A variant of ND/SK was also constructed which was predicted to lack Ig-binding activity. Each Ig-binding domain of protein L contains two Ig binding sites (designated site 1 and site 2), and residues critical to Ig binding at each site have been identified (7, 25, 31). Accordingly, the Ig-binding activity of site 1 was ablated by mutating residue 53 (Tyr to Phe) and residue 57 (Leu to His), and the Ig-binding activity of site 2 was ablated by mutating residue 66 (Val to Trp) using a PCR-based mutagenesis procedure. The resulting construct was designated pTRSB-E2S1-ND/SK(IBN), and the virus derived from this plasmid was designated IBN (immunoglobulin binding negative). Finally, sequences encoding the wild-type Ig-binding domain 1 were inserted downstream of E3 and fused directly to E2 (without the intervening linker sequence) using an overlapping PCR strategy. The amplicon produced in the final overlapping PCR reaction (containing the in frame contiguous sequences of capsid-E3-L1-E2) was digested with Aat II (in capsid sequence) and BssH II (in E2 sequence), and inserted into pTRSB-E2S1 from which a corresponding fragment had been removed. The resulting construct was designated pTRSB-E2S1-L1LN (L1 linker negative) and virus derived from this plasmid was designated L1LN.

A subset of the viruses was further modified to express the green fluorescent protein (GFP) from a duplicated 26S promoter placed into the 3′ non-translated region of the viral genome. Constructs encoding GFP-expressing versions of ND/SK and IBN were generated by transferring an Aat II/BssH II fragment from pTRSB-E2S1-ND/SK and pTRSB-E2S1-ND/SK(IBN), respectively, into pTRSB-E2S1-26S/GFP (39) from which a corresponding fragment had been removed. The resulting viruses were designated ND/SK-26S/GFP, and IBN-26S/GFP, respectively. The virus designated E2S1-GFP/2A expresses a GFP/2A fusion protein as a cleavable component of the viral structural polyprotein. This virus was constructed by transferring an Aat II/BssH II fragment from pTR339-GFP/2A (87) into pTRSB-E2S1 from which a corresponding fragment had been removed. All sequences that were generated and/or amplified by PCR during cloning procedures were confirmed by sequence analysis (Davis Sequencing, Davis, Calif.).

Infectious virus was derived from each cDNA clone as described previously (29, 45). Briefly, cDNA clones were linearized by digestion with Xho I, and run-off transcripts were produced using SP6 RNA polymerase. RNA transcripts where then electroporated into BHK-21 cells and virus containing growth medium was collected 24 hours post-electroporation and frozen at −80° C.

Virus Growth in BHK-21 and J774A.1 Cells.

The kinetics of virus growth was determined for selected viruses in BHK-21 cells. Cells were electroporated with in vitro viral transcripts as described above.

Electroporations were performed in duplicate for each virus and samples of growth medium were harvested at 6-hour intervals post-electroporation. Infectious virus was quantified by standard plaque assay on monolayers of BHK-21 cells. Virus titers were reported as the average of values obtained for the duplicate samples.

Subconfluent monolayers of J774A.1 cells were grown in 24-well plates (10⁶ cells/well). Prior to infection, viruses (2×10⁶ PFU/200 ul) were incubated under three different conditions. Virus was incubated alone, with normal mouse serum (1:40 dilution), or with heat-inactivated normal mouse serum (1:40 dilution). Medium was then aspirated from cells and 200 μl of each virus preparation was added to duplicate wells (multiplicity of infection=2). Virus was adsorbed to cells for 30 minutes at 37° C., and then cells were washed 3× with 1 ml of PBS containing 1% DCS and antibiotics. DMEM-complete was then added to each well and a sample was collected immediately (time point 0), and at 6-hour intervals thereafter. Infectious virus was quantified by standard plaque assay on monolayers of BHK-21 cells. Virus titers were reported as the average of values obtained for the duplicate samples.

Analysis of Radiolabeled Virions.

Virions were metabolically labeled with [³⁵S]-methionine during growth in BHK-21 cells essentially as described (29). Briefly, monolayers of BHK-21 cells were grown in 175 cm² flasks. Growth medium was removed from cells and infections were performed at a multiplicity of infection (MOI) of 1-5 PFU/cell. Virus was allowed to adsorb to cells for 30 minutes. Cells were washed 3× with phosphate buffered saline (PBS) to remove unbound virions and cells were then maintained in MEM-complete for 5 hours. Growth medium was then removed from cells and replaced with methionine-free MEM supplemented with 2% DCS, 10% TPB, and antibiotics. At 8 hours post-infection [35S]-methionine was added to a final concentration of 20 μCi/ml. Cells infected with L2, L3, and L4 were maintained for 28 hours after the addition of [³⁵S]-methionine. All other infections were maintained for 16 hours. After the labeling period, growth medium was harvested from each flask and clarified of cell debris by centrifugation (2500 RPM, 15 minutes, 4° C.). Clarified supernatants were then overlaid onto discontinuous potassium tartrate gradients (18% over 37%) made in TNE buffer (0.5M Tris-HCl, pH 7.2, 0.1M NaCl, 0.001M EDTA) and centrifuged at 24K RPM for 3 hours at 4° C. Virion-containing material that banded at the gradient interface was collected, diluted to 12 ml in TNE, overlaid onto 20% sucrose cushions (made in TNE), and centrifuged at 24K RPM for 3 hours at 4° C. Pelleted virions were harvested and quantified by liquid scintillation counting. Each virus preparation (100K CPM) was then resolved in SDS-polyacrylamide (10% acrylamide) gels and visualized by autoradiography.

Analysis of Virion Immunoglobulin Binding Properties.

Viruses were grown in BHK-21 cells and virions were purified from culture supernatants by ultracentrifugation as described above. ELISA plates (NUNC Maxisorp) were coated with purified viruses (100 ng/well) in carbonate buffer (pH 9.6) overnight at room temperature. Wells were washed 3× with PBS containing 0.1% Brij 35 (Sigma-Aldrich, St. Louis, Mo.) (PBS-Brij), then blocked for 1 hour with 3% bovine serum albumin in PBS (PBS-BSA). Wells were washed 3× with PBS-Brij and then 100 μl of biotinylated antibody (mouse IgG, human IgG, or goat IgG, Vector Laboratories, Burlingame, Calif.) was added to two adjacent top wells. These antibodies consisted of a 1:125 dilution of IgG stocks in PBS-BSA (concentrations of IgG stocks were 5 mg/ml [human and goat], or 1 mg/ml [mouse]). IgG samples were then processed in a 2-fold dilution series to a final dilution of 256K. Plates were incubated for 1 hr at room temperature and then plates were washed 6× with PBS-Brij. Streptavidin-horseradish peroxidase (1:500 dilution of 1 mg/ml stock) was then added to each well and incubated for 1 hour. Plates were then washed 4 times with PBS-Brij. 100 μl of substrate (o-phenylenediamine dihydrocholoride) was then added to each well, and optical density (OD₄₅₀) was measured 15 minutes later. The ELISA titer was calculated as the inverse of the IgG dilution that yielded OD_{450 nm} readings≧0.2 above background.

Immunoglobulin-Mediated Binding of Virions to Fc Receptor-Bearing Cells.

TRSB-E2S1-GFP/2A, NDSK-26S/GFP, and IBN-26S/GFP virions were incubated with serial 10-fold dilutions of normal mouse serum (untreated or heat-inactivated) in PBS and placed onto monolayers of J774A.1 cells at an MOI of 2 pfu/cell. Virus was allowed to adsorb to cells for 30 minutes at 37° C. Cells were then overlaid with MEM-complete. Cells were then viewed under a fluorescence microscope and infected cells were identified by GFP expression.

Recombinant Viruses Contain PpL/E2 Fusion Proteins and are Viable.

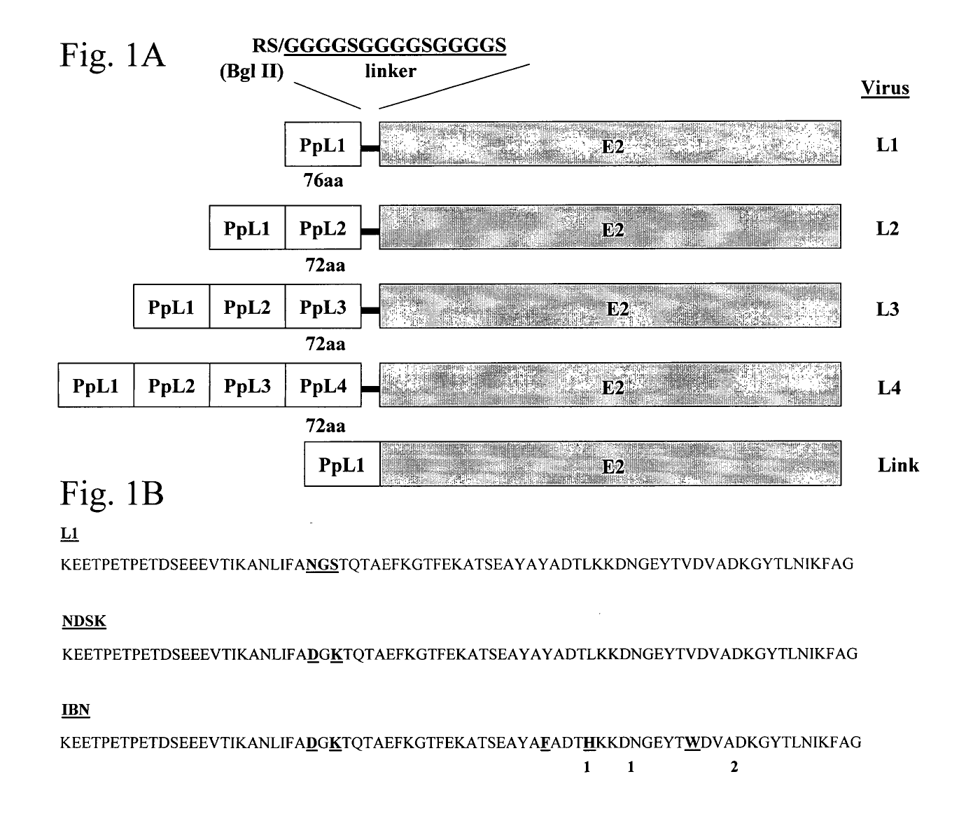

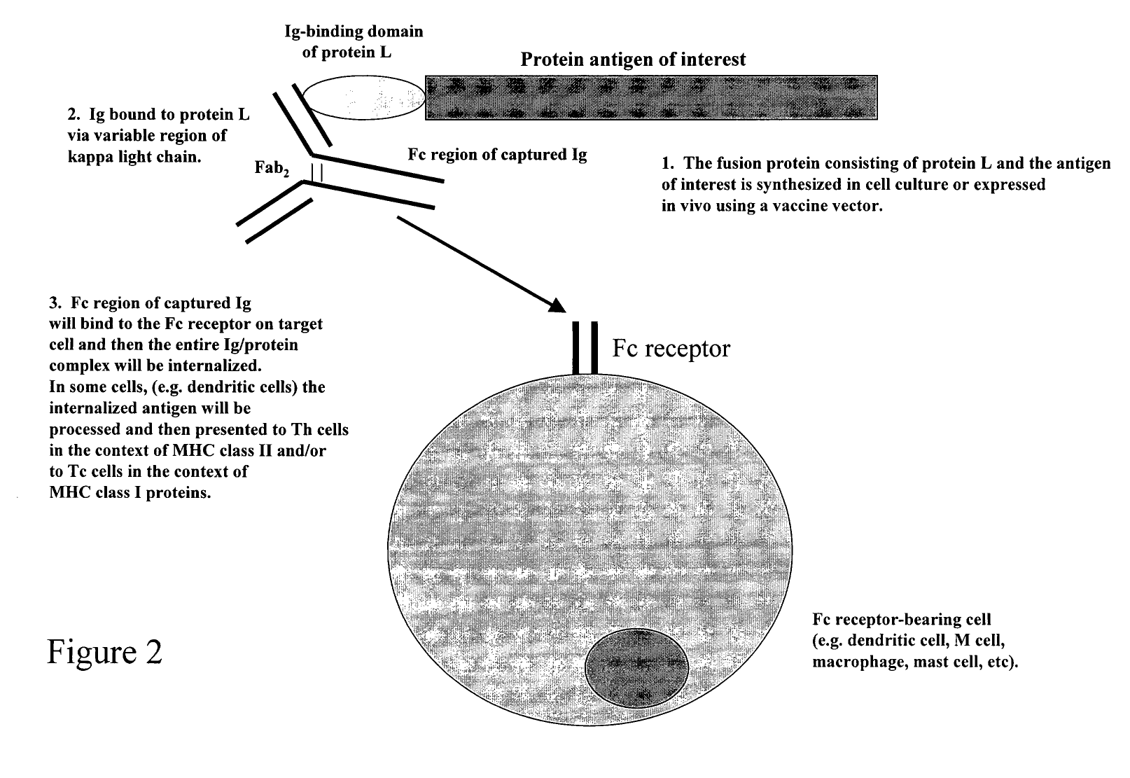

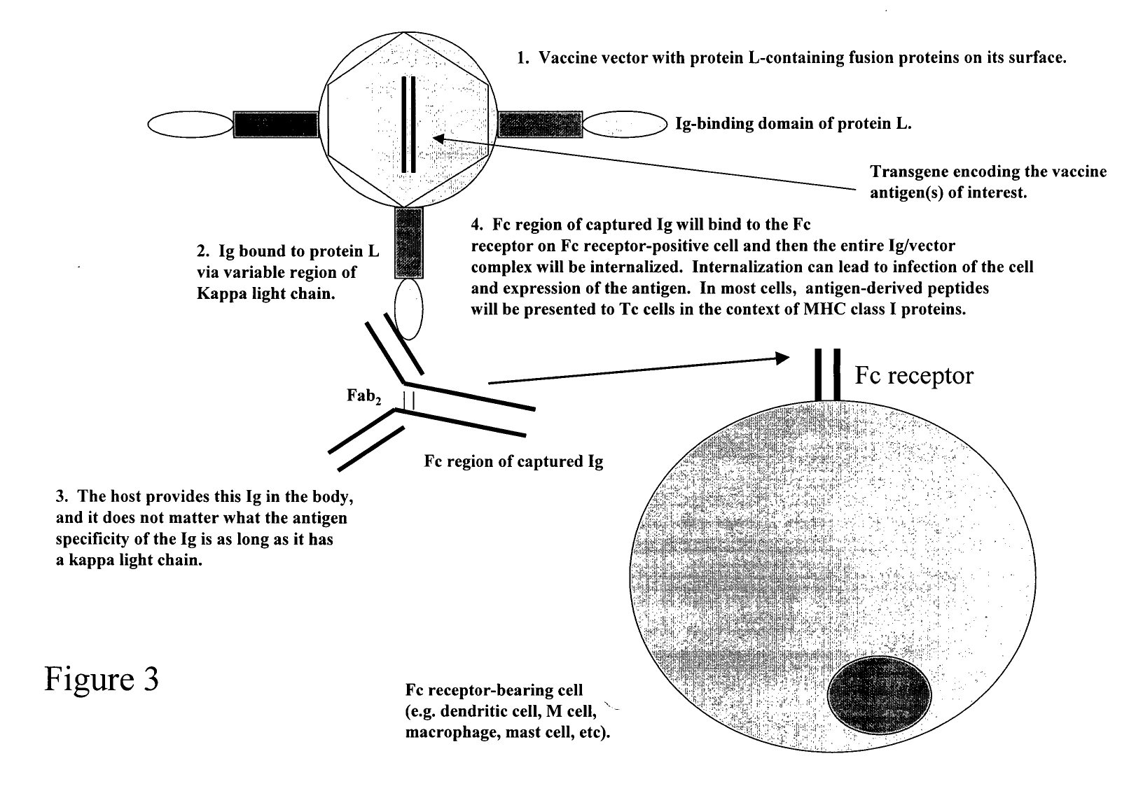

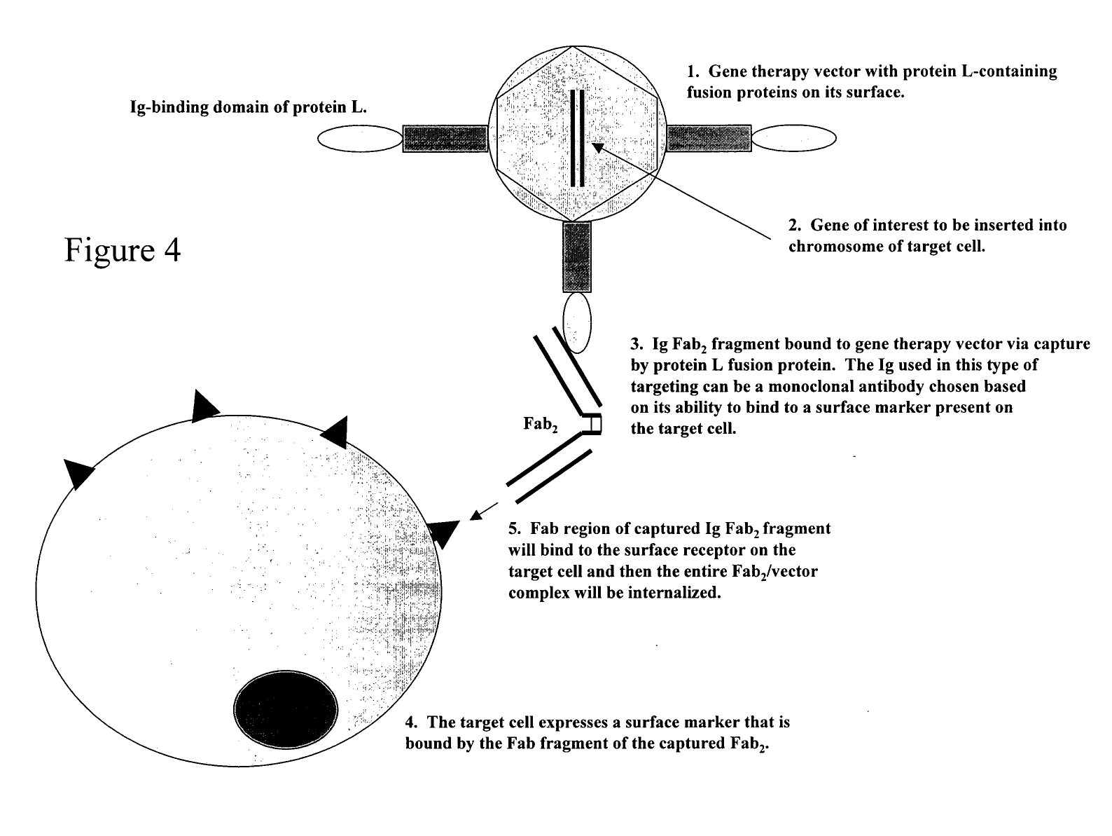

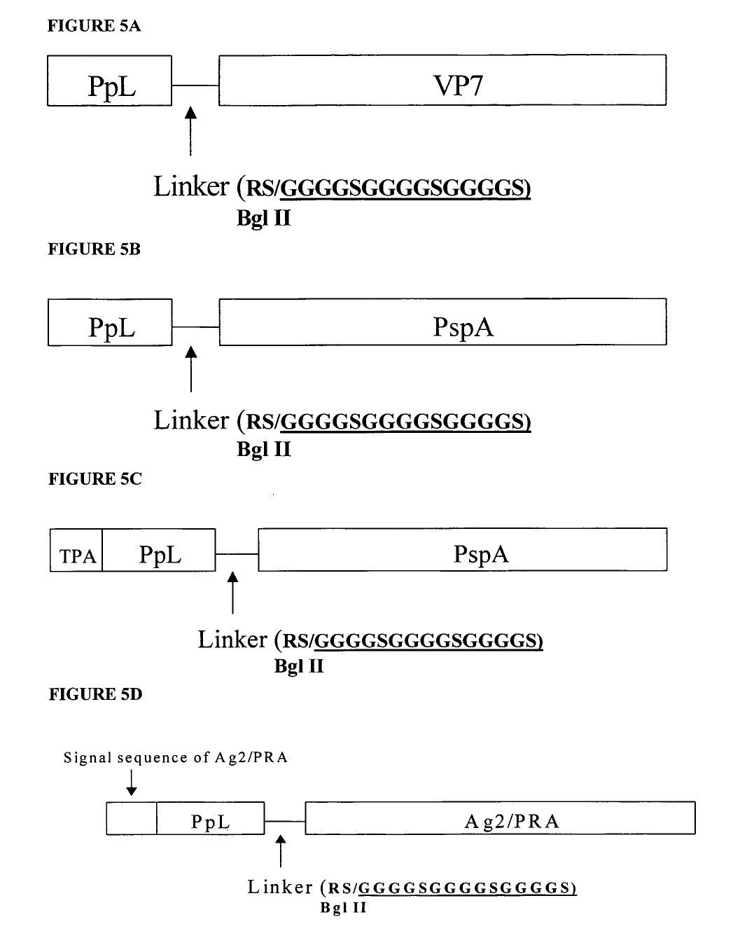

Recombinant Sindbis viruses were constructed which expressed PpL/E2 fusion proteins containing 1, 2, 3, or 4 Ig-binding domains of PpL as N-terminal extensions of E2 (FIG. 1A). These viruses were designated L1, L2, L3, and L4, respectively. The Ig binding domains were fused to E2 using a 17 amino acid linker element consisting of Arg-Ser (contributed by a Bgl II restriction site) followed by [Gly₄Ser]₃ (FIG. 1A). The core [Gly₄Ser]₃ element is believed to lack an ordered secondary structure and has been used successfully to link components of single chain antibodies which require proper folding to maintain function (33). Placement of this linker element between the PpL and E2 sequences would be expected to provide flexibility at the PpL/E2 junction, and flexibility at this site would facilitate proper folding of E2 and the establishment of functional interactions between E2 and E1. A virus containing a single Ig-binding domain fused directly to E2 (virus designated link-) was also constructed so that the contribution of the linker element to critical viral phenotypes could be assessed (FIG. 1A). Two additional variants of the L1 virus were also constructed. The first virus, designated ND/SK, is isogenic with L1 except for mutations at PpL residues 31 and 33, which were altered to eliminate a potential site for N-linked glycosylation (FIG. 1B). When expressed in bacteria, this site would not be expected to function as a glycosylation signal. However, when expressed in the context of a recombinant Sindbis virus this site could be glycosylated during virus replication in vertebrate cell lines and in vivo, and carbohydrate moieties bound at this site could potentially interfere with the Ig-binding properties of the protein. The second virus, designated ND/SK(Ab-) (also described as IBN), is isogenic with ND/SK except for two point mutations present within Ig-binding site 1, and a single mutation in Ig-binding site 2 (FIG. 1B). These mutations have been shown to independently ablate the Ig-binding activities of sites 1 and 2 (31), thus, ND/SK(Ab-) was expected to express the PpL Ig-binding domain 1 in a non-functional form.

The growth properties of the recombinant viruses were evaluated in BHK-21 cells. The viruses encoding a single PpL Ig-binding domain (L1, ND/SK, ND/SK(Ab-)/IBN and L1LN) grew at similar rates and achieved peak titers that were approximately 1 order of magnitude lower than that of E2S1. These viruses produced small plaques compared to E2S1 and this phenotype was maintained throughout the infection. The kinetics of virion production and the peak titer values determined for the L2, L3, and L4 viruses decreased progressively in accordance with the increasing number of PpL Ig-binding domains encoded by these viruses. L2 maintained a small plaque morphology throughout the 30-hour infection; however, the L3 and L4 viruses appeared to be unstable and steadily reverted to a large plaque phenotype.

Radiolabeled virions were then analyzed by SDS-PAGE to determine if the recombinant viruses incorporated PpL/E2 fusion proteins into their virion structure. Compared to the relative mobility (Mr) of E2 (expressed by E2S1), the Mr of the PpL/E2 fusion proteins encoded by L1, ND/SK, ND/SK(Ab-)/IBN, and Link- was decreased due to the incorporation of the single PpL Ig-binding domain. The Mr of the PpL/E2 protein of the Link- was increased slightly compared to that of L1. The subtle size difference observed between the PpL/E2 proteins of L1 and ND/SK suggests that the N-linked glycosylation site in L1 is utilized when virus is propagated in BHK-21 cells. The Mr of the PpL/E2 protein from L2 and L3 virions decreased in accordance with the increasing number of PpL Ig-binding domains encoded by these viruses. L4 virions did not appear to incorporate PpL/E2 fusion proteins containing the four PpL Ig-binding domains encoded by the virus. This result suggested that L4 was genetically unstable, and is consistent with the poor growth of L4 in BHK-21 cells and its rapid reversion to a large plaque phenotype.

To address this issue further, six large plaque variants of L4 were isolated on BHK-21 cells and plaque purified. Viral RNA was isolated from purified virions and the PpL sequences were amplified by RT-PCR and sequenced. Each virus contained a large in frame deletion within the PpL sequences resulting in the complete loss of Ig-binding domains 2 and 3, and most of domains 1 and 4. Consequently, no revertant virus encoded a PpL/E2 fusion protein containing more than 26 PpL-derived residues. Based on these results, the L4 virus was not studied further.

Recombinant Viruses Bind Ig in a Species-Specific Manner.

PpL binds Ig derived from many but not all mammalian species (15). For example, PpL binds with high affinity to kappa light chain-containing Igs derived from humans and mice, but does not bind Ig derived from goat and some other species. To determine if the recombinant viruses recapitulate the Ig-binding properties of native PpL, the ability of the recombinant viruses to bind Ig from various species was assessed by ELISA. Virions were grown in BHK-21 cells, purified by ultracentrifugation, and assayed for Ig-binding activity in ELISA. All recombinant viruses predicted to express functional PpL-derived Ig-binding domains (L1, L2, L3, ND/SK, and Link-), bound strongly to Ig from human and mouse, but bound weakly if at all to Ig from goat. Binding of human and mouse Ig by these viruses was specific, as Ig-binding was not detected in wells coated with the parental virus, E2S1, or in wells that contained no viral antigens. In addition, binding of human and mouse Ig by the recombinant viruses was mediated by the PpL component of the viral spike as ND/SK(Ab-) failed to bind Ig above background levels.

Ig-Binding Viruses Display ADE of Infection of FcR-Positive Cells.

J774A.1 cells are murine monocyte/macrophage-like cells that express high and low affinity FcγRs on their surface (14, 63) and efficiently endocytose IgG-opsonized microbial agents (4, 53). J774A.1 cells are susceptible to ADE of infection by some viruses (23, 92), suggesting that FcγR-mediated internalization of virus/Ig complexes into J774A.1 cells can lead to productive virus infection. J774A.1 cells were shown to be nearly refractory to Sindbis virus infection following incubation with virions. In the absence of murine Ig, virus titers produced by J774A.1 cultures exposed to E2S1, ND/SK, or ND/SK(ab-) virions increased only slightly above background levels (virus present at time 0). This slight increase in virus titer probably reflects virus produced by the small minority of cells (% to %) that were shown to support viral gene expression following infection with the GFP-expressing versions of these viruses. Incubation of E2S1 and ND/SK(ab-) virions with normal mouse serum had a minimal effect on infection of J774A.1 cells as this treatment did not result in significantly enhanced viral titers following infection, or increased numbers of GFP-expressing cells. In contrast, incubation of ND/SK virions with normal mouse serum enhanced the infectivity of the virions for J774A.1 cells, as demonstrated by marked increases in virus titer, and percentage of GFP-expressing cells. Similar results were obtained when mouse serum was heat inactivated prior to incubation with virions, suggesting that the enhancement of ND/SK infectivity was mediated directly by the Ig component of the serum, and not by complement components.

### Example 2

Methods: Two CD-1 outbred mice, 3-5 weeks of age, received a 10 microliter inoculation in both rear footpads of either undiluted, 1:10-diluted or 1:100-diluted (diluent: phosphate buffered saline with 1% donor calf serum) ND/SKGFP Sindbis virus. At eight hours post inoculation, mice were euthanized and the draining popliteal lymph nodes (DLN) were harvested (4 lymph nodes). Expression of green fluorescent protein (GFP), an indicator of successful infection and Sindbis virus gene expression, was assessed on a Nikon TE300 fluorescence microscope using an Endow GFP filter. This experiment was repeated two times.

Results: Lymph nodes from mice inoculated with all dilutions of the Protein L-fusion virus (ND/SK) exhibited GFP-expressing cells in the subcapsular sinus. These cells were provisionally identified as dendritic cells and/or marginal zone macrophages by their morphology and location.

### Example 3

Methods: Undiluted ND/SKGFP virus or E2S12AGFP control virus was reacted (30 minutes at 4 C.) with a 1:1000 dilution (diluent: phosphate buffered saline with 1% donor calf serum) of either human serum or mouse serum (appropriate to the species of the Fc receptor-bearing cell) and either goat serum or serum-free reaction as a control. Subsequently, reaction mixtures were incubated (30 minutes at 37 C) with the following Fc receptor-expressing cells: mouse J774 and Raw264.7 macrophages, human THP-1 pre-myelocytes and human Raji B cells. After this incubation, cells were washed three times with diluent, growth medium was replaced and cells were incubated for 12-14 hours followed by observation for green fluorescent protein (GFP) as an indication of virus infection and gene expression.

Results: In each case, the 1:1000 dilution of species-appropriate human or mouse serum significantly increased infection of the cells (estimated to be more than two orders of magnitude increase) by the ND/SK virus while goat serum or serum-free reactions exhibited little to no effect and none of the sera had any effect upon the infectivity of the E2S12AGFP control virus. In the case of the J774, THP-1 and Raji cells, the E2S12AGFP control virus infected a very small percentage of the cells regardless of treatment while the ND/SK virus incubated with the species-appropriate serum appeared to infect most cells. Thus, the Protein L-Fc receptor entry mechanism appears to have the capacity to overcome infection resistance resulting from limited expression of natural Sindbis virus attachment and entry receptors.

### Example 4

Construction of Protein L-Containing Vaccine Antigens.

The following three antigens have been modified as fusion proteins with a single Ig-binding domain of protein L. All recombinant genes have been engineered to be expressed from a duplicated 26S promoter placed within the 3′ non-translated region of Sindbis virus strain TRSB-E2S1.

1. VP7 Core Protein of Bluetongue Virus Serotype 10 (SEQ ID NOs:22/23) (Provided by Dr. William Wilson of the University of Wyoming at Laramie).

PpL/VP7 fusion proteins have been produced that contain the ND/SK (non-glycosylated) and the IBN (Ig-binding negative) versions of PpL binding domain #1. The PpL sequences represent the N-terminal segment of the fusion protein and are linked to the downstream VP7 sequences through a 15 amino acid linker segment (FIG. 5A).

2. PspA (Pneumococcal Surface Protein A) Protein of Streptococcus pneumoniae (SEQ ID NOs:26/27 (Provided by Dr. David Britles of the University of Alabama at Birmingham)

PpL/PspA fusion proteins have been produced that contain the ND/SK and IBN version of versions of PpL binding domain #1. The PpL sequences represent the N-terminal segment of the fusion protein and are linked to the downstream PspA sequences through a 15 amino acid linker segment (FIG. 5B).

Two additional versions of the PpL/PspA fusion protein have been constructed in which the signal sequence of the human tissue plasminogen activating factor (TPA) has been fused upstream of the PpL sequences (ND/SK and IBN versions) (FIG. 5C).

3. Ag2/PRA (Antigen 2/Proline Rich Antigen) Protein of Coccidiodes immitis (SEQ ID NOs:24/25) (Provided by Dr. Mitchell Magee of the University of Texas Health Sciences Center at San Antonio).

PpL/Ag2/PRA fusion proteins have been produced that contain the ND/SK and IBN version of PpL binding domain #1. The PpL sequences have been inserted internally within the Ag2/PRA sequences between the signal sequence and downstream regions of Ag2/PRA. The PpL sequences are linked to the downstream Ag2/PRA sequences through a 15 amino acid linker segment (FIG. 5D).

To test these fusion proteins, a blood sample will be collected from mice prior to vaccination. Recombinant viruses engineered to produce these fusion proteins will then be administered to mice by subcutaneous or intranasal inoculation. Mice will be boosted with recombinant virus at 21 and 42 days. At 56 days, a blood sample will be collected, separated into cell and serum fractions, and serum will be frozen. Serum will be assayed by ELISA to detect and quantify antigen-specific antibodies. Spleens will be harvested from vaccinated mice and antigen-specific T cell responses will be assessed using T cell recall assays, cytokine-specific RT-PCR, or ELISPOT assays.

The foregoing is considered as illustrative only of the principles of the invention. Further, since numerous modifications and changes will readily occur to those skilled in the art, it is not desired to limit the invention to the exact construction and operation shown and described herein. Therefore, accordingly, all suitable modifications and equivalents fall within the scope of the invention.

All publications, patent applications, patents and other references cited herein are incorporated by reference in their entireties for the teachings relevant to the sentence and/or paragraph in which the reference is presented.

### REFERENCES

1. Akerstrom, B., and L. Bjorck. 1989. Protein L: An immunoglobulin light chain-binding bacterial protein. J. Biol. Chem. 264:19740-19746.
3. Amigorena, S. 2002. Fcg receptors and cross-presentation in dendritic cells. J. Exp. Med. 195:F1-F3.
4. Arulanandam, B. P., J. M. Lynch, D. E. Briles, S. Hollingshead, and D. W. Metzger. 2001. Intranasal vaccination with Pneumococcal surface protein A and interleukin-12 augments antibody-mediated opsonization and protective immunity against Streptococcus pneumoniae infection. Infect. Immun. 69:6718-6724.
5. Banchereau, J., and R. M. Steinman. 1998. Dendritic cells and the control of immunity. Nature 392:245-252.
6. Bashirova, A. A., T. B. Geijtenbeek, G. C. van Duijnhoven, S. J. Van Vliet, J. B. Eilering, M. P. Martin, L. Wu, T. D. Martin, N. Viebig, P. A. Knolle, V. N. KewalRamani, Y. van Kooyk, and M. Carrington. 2001. A dendritic cell-specific intercellular adhesion molecule 3-grabbing nonintegrin (DC-SIGN)-related protein is highly expressed on human liver sinusoidal endothelial cells and promotes HIV-1 infection. J. Exp. Med. 193:671-678.
7. Beckingham, J. A., H. R. Housden, N. M. Muir, S. P. Bottomley, and M. G. Gore. 2001. Studies on a single immunoglobulin-binding domain of protein L from Peptostreptococcus magnus: the role of tyrosine-53 in the reaction with human IgG. Biochem. J. 353:395-401.
10. Bjorck, L. 1988. Protein L: a novel bacterial cell protein with affinity for Ig chains. J. Immunol. 140:1194-1197.
11. Bjorck, L., and G. Kronvall. 1984. Purification and some properties of streptococcal protein G, a novel IgG-binding reagent. J. Immunol. 133:969-974.
14. Cote-Velez, M. J. A., E. Ortega, and A. Ortega. 2001. Involvement of pp125^{FAK} and p60^{SRC} in the signaling through FCgRII-FcgRIII in murine macrophages. Immunology Letters 78:189-194.
15. De Chateau, M., B. H. K. Nilson, M. Erntell, E. Myhre, C. G. M. Magnusson, B. Akerstrom, and L. Bjorck. 1993. On the interaction between protein L and immunoglobulins of various mammalian species. Scand. J. Immunol. 37:399-405.
18. Enokizono, J., M. Wikstrom, U. Sjobring, L. Bjorck, S. Forsen, Y. Arata, K. Kato, and I. Shimada. 1997. NMR analysis of the interaction between protein L and Ig light chains. J. Mol. Biol. 270:8-13.
19. Forsgren, A., and J. Sjoquist. 1966. “Protein A” from Staphylococcus aureus. I. Pseudo-immune reaction with human gamma-globulin. J. Immunol. 97:822-827.
20. Frolov, I., T. A. Hoffman, B. M. Pragai, S. A. Dryga, H. V. Huang, S. Schlesinger, and C. M. Rice. 1996. Alphavirus-based expression vectors: Strategies and applications. Proc. Natl. Acad. Sci. U.S.A. 93:11371-11377.
21. Gardner, J. P., I. Frolov, S. Perri, Y. Ji, M. L. MacKichan, J. Z. Megede, M. Chen, B. A. Belli, D. A. Driver, S. Sherrill, C. E. Greer, G. R. Otten, S. W. Barnett, M. A. Liu, T. W. Dubensky, and J. M. Polo. 2000. Infection of human dendritic cells by a Sindbis virus replicon vector is determined by a single amino acid substitution in the E2 glycoprotein. J. Virol. 74:11849-11857.
23. Girn, J., M. Kavoosi, and J. Chantler. 2002. Enhancement of coxsackievirus B3 infection by antibody to a different coxsackievirus strain. J. Gen. Virol. 83:351-358.
24. Graille, M., S. Harrison, M. P. Crump, S. C. Findlow, N. G. Housden, B. H. Muller, N. Battail-Poirot, G. Sibai, B. J. Sutton, M. J. Taussig, C. Jolivet-Reynaud, M. G. Gore, and E. A. Stura. 2002. Evidence for plasticity and structural mimicry at the immunoglobulin light chain-protein interface. J. Biol. Chem. 277:47500-47506.
25. Graille, M., E. A. Stura, N. G. Housden, J. A. Beckingham, S. P. Bottomley, D. Beale, M. J. Taussig, B. J. Sutton, M. G. Gore, and J.-B. Charbonnier. 2001. Complex between Peptostreptococcus magnus protein L and a human antibody reveals structural convergence in the interaction modes of Fab binding proteins. Structure 9:679-687.
26. Guermonprez, P., J. Valladeau, L. Zitvogel, C. Thery, and S. Amigorena. 2002. Antigen presentation and T cell stimulation by dendritic cells. Annu. Rev. Immunol. 20:621-667.
28. Heidner, H. W., and R. E. Johnston. 1994. The amino-terminal residue of Sindbis virus glycoprotein E2 influences virus maturation, specific infectivity for BHK cells, and virulence in mice. J. Virol. 68:8064-8070.
29. Heidner, H. W., K. L. McKnight, N. L. Davis, and R. E. Johnston. 1994. Lethality of PE2 incorporation into Sindbis virus can be suppressed by second-site mutations in E3 and E2. J. Virol. 68:2683-2692.
31. Housden, N. G., S. Harrison, H. R. Housden, K.-A. Thomas, J. A. Beckingham, S. E. Roberts, S. P. Bottomley, M. Graille, E. Stura, and M. G. Gore. 2004. Observation and characterization of the interface between a single immunoglobulin binding domain of protein L and two human kappa light chains. J. Biol. Chem.
32. Hsieh, P., and P. W. Robbins. 1984. Regulation of asparagine-linked olgosaccharide processing. Oligosaccharide processing in Aedes albopictus mosquito cells. J. Biol. Chem. 259:2375-2382.
33. Huston, J. S., D. Levinson, M. Medgett-Hunter, M.-S. Tai, J. Novotny, M. N. Margolies, R. J. Ridge, R. E. Bruccoleri, E. Haber, R. Crea, and H. Opperman. 1988. Protein engineering of antibody binding sites: recovery of specific activity in an anti-digoxin single-chain Fv analogue produced in Escherichia coli. Proc. Natl. Acad. Sci. U.S.A. 85:5879-5883.
36. Kastern, W., E. Holst, E. Nielsen, U. Sjobring, and L. Bjorck. 1990. Protein L, a bacterial immunoglobulin-binding protein and a possible virulence determinant. Infect. Immun. 58:1217-1222.
37. Kastern, W., U. Sjobring, and L. Bjorck. 1992. Structure of Peptostreptococcal protein L and identification of a repeated immunoglobulin light chain-binding domain. J. Biol. Chem. 267:12820-12825.
39. Klimstra, W. B., E. M. Nangle, M. S. Smith, A. D. Yurochko, and K. D. Ryman. 2003. DC-SIGN and L-SIGN can act as attachment receptors for alphaviruses and distinguish between mosquito cell- and mammalian cll-derived viruses. J. Virol. 77.
40. Klimstra, W. B., K. D. Ryman, K. A. Bernard, K. B. Nguyen, C. A. Biron, and R. E. Johnston. 1999. Infection of neonatal mice with Sindbis virus results in a systemic inflammatory response syndrome. J. Virol. 73:10387-10398.
41. Klimstra, W. B., K. D. Ryman, and R. E. Johnston. 1998. Adaptation of Sindbis virus to BHK cells selects for use of heparan sulfate as an attachment receptor. J. Virol. 72:7357-7366.
42. Krasnykh, V., J. T. Douglas, and V. W. van Beusechem. 2000. Genetic targeting of adenoviral vectors. Mol. Ther. 1:391-405.
43. Kuhn, R. J., D. E. Griffin, K. E. Owen, H. G. Niesters, and J. H. Strauss. 1996. Chimeric Sindbis-Ross River viruses to study interactions between alphavirus nonstructural and structural proteins. J. Virol. 70:7900-7909.
45. Liljestrom, P., S. Lusa, D. Huylebroeck, and H. Garoff. 1991. In vitro mutagenesis of a full-length cDNA clone of Semliki Forest virus: the small 6,000-molecular weight membrane protein modulates virus release. J. Virol. 65:4107-4113.
50. McKnight, K. L., D. A. Simpson, S. C. Lin, T. A. Knott, J. M. Polo, D. F. Pence, D. B. Johannsen, H. W. Heidner, N. L. Davis, and R. E. Johnston. 1996. Deduced consensus sequence of Sindbis virus strain AR339: mutations contained in laboratory strains which affect cell culture and in vivo phenotypes. J. Virol. 70:1981-1989.
51. Moll, H. 2003. Dendritic cells and host resistance to infection. Cell. Microbiol. 5:493-500.
53. Mukherjee, S., S. C. Lee, and A. Casadevall. 1995. Antibodies to Cryptococcus neoformans glucuronoxylomannan enhance antifungal activity of murine macrophages. Infect. Immun. 63:573-579.
54. Murphy, J. P., C. J. Duggleby, M. A. Atkinson, A. R. Trowern, T. Atkinson, and C. R. Goward. 1994. The functional units of a peptostreptococcal protein L. Mol. Microbiol. 12:911-920.
55. Myhre, E., and M. Erntell. 1985. A non-immune interaction between the light chain of immunoglobulin and a surface component of Peptostreptococcus magnus strain.
57. Nilson, B. H. K., L. Logdberg, W. Kastern, L. Bjorck, and B. Akerstrom. 1993. Purification of antibodies using protein L-binding framework structures in the light chain variable domain. J. Immunol. Methods 164:33-40.
58. Nilson, B. H. K., A. Solomon, L. Bjorck, and B. Akerstrom. 1992. Protein L from Peptostreptococcus magnus binds to the k light chain variable domain. J. Biol. Chem. 267:2234-2239.
60. Paessler, S., R. Z. Fayzulin, M. Anishchenko, I. P. Greene, S. C. Weaver, and I. Frolov. 2003. Recombinant Sindbis/Venezuelan equine encephalitis virus is highly attenuated and immunogenic. J. Virol. 77:9278-9286.
63. Ralph, P., J. Prichard, and M. Cohn. 1975. Reticulum cell sarcoma: an effector cell in antibody-dependent cell-mediated immunity. J. Immunol. 114:898-905.
64. Rayner, J. O., S. A. Dryga, and K. I. Kamrud. 2002. Alphavirus vectors and vaccination. Rev. Med. Virol. 12:279-296.
65. Regnault, A., D. Lankar, V. Lacabanne, A. Rodriguez, C. Thery, M. Rescigno, T. Saito, S. Verbeek, C. Bonnerot, P. Ricciardi-Castagnoli, and S. Amigorena. 1999. Fcg receptor-mediated induction of dendritic cell maturation and major histocompatibility complex class I-restricted antigen presentation after immune complex internalization. J. Exp. Med. 189:371-380.
66. Reis, K., E. Ayoub, and M. Boyle. 1984. Streptococcal Fc receptors. I. Isolation and partial characterization of the receptor from group C streptococcus. J. Immunol. 132:3091-3097.
67. Ricci, S., D. Medaglini, H. Marcotte, A. Olsen, G. Pozzi, and L. Bjorck. 2001. Immunoglobulin-binding domains of peptostreptococcal protein L enhance vaginal colonization of mice by Streptococcus gordonii. Microb. Pathog. 30:229-235.
69. Roks, A. J., R. H. Henning, H. Buikema, Y. M. Pinto, M. J. Kraak, R. A. Tio, D. de Zeeuw, H. J. Haisma, J. Wilschut, and W. H. van Gilst. 2002. Recombinant Semliki Forest virus as a vector system for fast and selective in vivo gene delivery into balloon-injured rat aorta. Gene Ther. 9:95-101.
70. Ryman, K. D., W. B. Klimstra, K. B. Nguyen, C. A. Biron, and R. E. Johnston. 2000. Alpha/beta interferon protects adult mice from fatal Sindbis virus infection and is an important determinant of cell and tissue tropism. J. Virol.
71. Ryman, K. D., L. J. White, R. E. Johnston, and W. B. Klimstra. 2002. Effects of PKR/RNase-L-dependent and alternative antiviral pathways on alphavirus replication and pathogenesis. Viral Imm. 15:53-76.
72. Sandrin, V., S. J. Russell, and F. L. Cosset. 2003. Targeting retroviral and lentiviral vectors. Curr. Top. Microbial. Immunol. 281:137-78.
74. Schlesinger, S. 2000. Alphavirus expression vectors. Adv. Virus Res. 55:565-577.
75. Schlesinger, S., and T. W. Dubensky. 1999. Alphavirus vectors for gene expression and vaccines. Curr. Opin. Biotechnol. 10:434-439.
76. Schoepp, R. J., J. F. Smith, and M. D. Parker. 2002. Recombinant chimeric Western and Eastern equine encephalitis viruses as potential vaccine candidates. Virology 302:299-309.
77. Schuurhuis, D. H., A. Ioan-Facsinay, B. Nagelkerken, J. J. van Schip, C. Sedlik, C. J. M. Melief, J. S. Verbeek, and F. Ossendorp. 2002. Antigen-antibody immune complexes empower dendritic cells to efficiently prime specific CD8+ CTL responses in vivo. J. Immunol. 168:2240-2246.
80. Smyth, J., M. Suomalainen, and H. Garoff. 1997. Efficient multiplication of a Semliki Forest virus chimera containing Sindbis virus spikes. J. Virol. 71:818-823.
81. Soilleux, E. J., S. L. Morris, G. Leslie, J. Chehimi, Q. Luo, E. Levroney, J. Trowsdale, L. J. Montaner, R. W. Doms, D. Weissman, N. Coleman, and B. Lee. 2002. Constitutive and induced expression of DC-SIGN on dendritic cell and macrophage subpopulations in situ and in vitro. J. Leukoc. Biol. 71:445-457.
82. Solomon, A. N. 1976. Bence-Jones proteins and light chains of immunoglobulins. New Engl. J. Med. 294:17-23.
83. Steinman, R. M., and M. Pope. 2002. Exploiting dendritic cells to improve vaccine efficacy. J. Clin. Invest. 109:1519-1526.
84. Strauss, J. H., and E. G. Strauss. 1994. The alphaviruses: gene expression, replication, and evolution. Microbiol. Rev. 58:491-562.
85. Sullivan, K. F. 1999. Enlightening mitosis: construction and expression of green fluorescent fusion proteins. Meth. Cell Biol. 61:113-135.
87. Thomas, J. M., W. B. Klimstra, K. D. Ryman, and H. W. Heidner. 2003. Sindbis virus vectors designed to express a foreign protein as a cleavable component of the viral structural polyprotein. J. Virol. 77:5598-5606.
91. Wickham, T. J. 2003. Ligand-directed targeting of genes to the site of disease. Nature Med. 9:135-139.
92. Yao, J. S., H. Kariwa, I. Takashima, K. Yoshimatsu, J. Arikawa, and N. Hashimoto. 1992. Antibody-dependent enhancement of hantavirus infection in macrophage cell lines. Arch. Virol. 122:107-118.
93. Yao, J. S., E. G. Strauss, and J. H. Strauss. 1998. Molecular genetic study of the interaction of Sindbis virus E2 with Ross River virus E1 for virus budding. J. Virol. 72:1418-1423.

1. A fusion protein comprising a first amino acid sequence of at least one immunoglobulin-binding domain of Protein L and a second amino acid sequence of a peptide or protein that does not bind an immunoglobulin Fc region.

2. The fusion protein of claim 1, wherein the peptide or protein that does not bind an immunoglobulin Fc region is a peptide or protein of Protein A, Protein G, Protein H or Protein M.

3. The fusion protein of claim 1, comprising the amino acid sequences oftwo immunoglobulin binding domains of Protein L.

4. The fusion protein of claim 1, comprising the amino acid sequences of three immunoglobulin binding domains of Protein L.

5. The fusion protein of claim 1, comprising the amino acid sequences of four immunoglobulin binding domains of Protein L.

6. The fusion protein of claim 1, comprising the amino acid sequences of five immunoglobulin binding domains of Protein L.

7. The fusion protein of claim 1, wherein the first amino acid sequence is joined to the second amino acid sequence by a linker amino acid sequence.

8. The fusion protein of claim 7, wherein the linker amino acid sequence comprises at least three amino acids.

9. The fusion protein of claim 7, wherein the linker amino acid sequence comprises at least ten amino acids.

10. The fusion protein of claim 7, wherein the linker amino acid sequence comprises (Gly-Gly-Gly-Gly-Ser)₃.

11. The fusion protein of claim 1, wherein the second amino acid sequence is an amino acid sequence of a virus protein.

12. The fusion protein of claim 11, wherein the virus protein is an alphavirus E2 protein.

13. The fusion protein of claim 1, wherein the second amino acid sequence is an amino acid sequence of an immunogenic peptide.

14. The fusion protein of claim 1, wherein the second amino acid sequence is an amino acid sequence of a vector comprising a nucleic acid encoding an immunogenic or therapeutic protein or peptide.

15. A composition comprising the fusion protein of claim 1, complexed with an immunoglobulin molecule or a Fab₂ fragment of an immunoglobulin molecule.

16. A nucleic acid encoding the fusion protein of claim 1.

17. A vector comprising the nucleic acid of claim 16.

18. A cell comprising the vector of claim 17.

19. A virus particle comprising the fusion protein of claim 11.

20. An alphavirus particle comprising the fusion protein of claim 12.

21. A method of making a fusion protein comprising a first amino acid sequence of at least one immunoglobulin-binding domain of Protein L and a second amino acid sequence of a peptide or protein that does not bind an immunoglobulin Fc region, comprising:
a) culturing cells comprising a recombinant nucleic acid encoding a fusion protein comprising a first amino acid sequence of at least one immunoglobulin-binding domain of Protein L and a second amino acid sequence of a peptide or protein that does not bind an immunoglobulin Fc region under conditions whereby the recombinant nucleic acid is expressed to produce the fusion protein; and
b) collecting the fusion protein from the cells.

22. A method of delivering a fusion protein to an Fc receptor-bearing cell of a subject, comprising administering to the subject an effective amount of the fusion protein of claim 1.

23. A method of delivering a fusion protein to an Fc-receptor bearing cell of a subject, comprising administering to the subject an effective amount of the composition of claim 15.

24. A method of delivering a therapeutic or immunogenic protein or peptide to an Fc-bearing receptor cell in a subject, comprising administering to the subject an effective amount of the fusion protein of claim 14.

25. A method of delivering a therapeutic or immunogenic substance to a target cell in a subject, comprising administering to the subject an effective amount of a composition comprising:
a) a fusion protein comprising a first amino acid sequence of at least one immunoglobulin-binding domain of Protein L and a second amino acid sequence of a therapeutic or immunogenic protein or peptide; and
b) an Fab₂ fragment of an antibody specific for a receptor on the surface of the target cell.

26. A method of delivering a therapeutic or immunogenic substance to a target cell in a subject, comprising administering to the subject an effective amount of a composition comprising:
a) a fusion protein comprising a first amino acid sequence of at least one immunoglobulin-binding domain of Protein L and a second amino acid sequence which is an amino acid sequence of a vector comprising a nucleic acid encoding an immunogenic or therapeutic protein or peptide; and
b) an Fab₂ fragment of an antibody specific for a receptor on the surface of the target cell.

27. A method of eliciting an immune response in a subject, comprising administering to the subject an effective amount of a composition comprising:
a) a fusion protein comprising a first amino acid sequence of at least one immunoglobulin-binding domain of Protein L and a second amino acid sequence of an immunogenic protein or peptide; and
b) an Fab₂ fragment of an immunoglobulin molecule specific for a receptor on the surface of the target cell or an immunoglobulin molecule capable of binding an Fc receptor on a cell.

28. A method of eliciting an immune response in a subject, comprising administering to the subject an effective amount of a composition comprising:
a) a fusion protein comprising a first amino acid sequence of at least one immunoglobulin-binding domain of Protein L and a second amino acid sequence which is an amino acid sequence of a vector comprising a nucleic acid encoding an immunogenic protein or peptide; and
b) an Fab₂ fragment of an immunoglobulin molecule specific for a receptor on the surface of the target cell or an immunoglobulin molecule capable of binding an Fc receptor on a cell.

29. A method of treating cancer in a subject in need thereof, comprising administering to the subject an effective amount of a composition comprising:
a) a fusion protein comprising a first amino acid sequence of at least one immunoglobulin binding domain of Protein L and a second amino acid sequence of a substance that is toxic to the cancer cell; and
b) an Fab₂ fragment of an antibody specific for a receptor on the surface of a cancer cell of the subject.

30. A method of treating cancer in a subject in need thereof, comprising administering to the subject an effective amount of a composition comprising:
a) a fusion protein comprising a first amino acid sequence of at least one immunoglobulin binding domain of Protein L and a second amino acid sequence which is an amino acid sequence of a vector comprising a nucleic acid encoding a substance that is toxic to the cancer cell; and
b) an Fab₂ fragment of an antibody specific for a receptor on the surface of a cancer cell of the subject.

31. A method of treating cancer in a subject in need thereof, comprising administering to the subject an effective amount of a composition comprising:
a) a fusion protein comprising a first amino acid sequence of at least one immunoglobulin binding domain of Protein L and a second amino acid sequence which is an amino acid sequence of an oncolytic virus; and
b) an Fab₂ fragment of an antibody specific for a receptor on the surface of a cancer cell of the subject.

32. The method of claim 31, wherein the oncolytic virus is an alphavirus.

33. A pharmaceutical composition comprising the fusion protein of claim 1 in a pharmaceutically acceptable carrier.

34. A pharmaceutical composition comprising the composition of claim 15 in a pharmaceutically acceptable carrier.

35. A pharmaceutical composition comprising the vector of claim 17 in a pharmaceutically acceptable carrier.

36. A pharmaceutical composition comprising the virus particle of claim 19 in a pharmaceutically acceptable carrier.
